(19) United States
(12) Patent Application Publication
(10) Pub. No.: US 2023/0277519 A1
(43) Pub. Date: Sep. 7, 2023

(54) IRAK4 DEGRADERS AND USES THEREOF

(71) Applicant: Kymera Therapeutics, Inc., Watertown, MA (US)

(72) Inventors: Jared Gollob, Watertown, MA (US); Jeffrey Davis, Watertown, MA (US); Ashwin Gollerkeri, Watertown, MA (US); Reginald EWESUEDO, Watertown, MA (US); Alice McDonald, Watertown, MA (US); Vashali Dixit, Watertown, MA (US); Shu-Pei Wu, Watertown, MA (US); Michele Mayo, Watertown, MA (US); Haojing Rong, Watertown, MA (US); Sagar Agarwal, Watertown, MA (US); Bradley Enerson, Watertown, MA (US); Patrick Henrick, Watertown, MA (US); Rachelle Perea, Watertown, MA (US); Christopher Ho, Watertown, MA (US); William Leong, Watertown, MA (US); Duncan Walker, Watertown, MA (US)

(21) Appl. No.: 18/154,454

(22) Filed: Jan. 13, 2023

Related U.S. Application Data
(60) Provisional application No. 63/387,274, filed on Dec. 13, 2022.
(60) Provisional application No. 63/267,465, filed on Feb. 2, 2022.
(60) Provisional application No. 63/299,653, filed on Jan. 14, 2022.

(51) Int. Cl. A61K 31/4545; A61P 35/00

(52) U.S. Cl. CPC A61K 31/4545; A61P 35/00

(57) ABSTRACT

The present invention relates to IRAKIMiD degraders, their liquid formulations, and methods of use thereof for treating cancer.

### CROSS-REFERENCE TO RELATED APPLICATIONS

This application claims the benefit of priority to U.S. Provisional Appl. No. 63/299,653, filed Jan. 14, 2022, U.S. Provisional Appl. No. 63/267,465, filed Feb. 2, 2022, and U.S. Provisional Appl. No. 63/387,274, filed Dec. 13, 2022, the content of each of which is herein incorporated by reference.

### TECHNICAL FIELD OF THE INVENTION

The present invention relates to formulation and dosage forms of IRAKIMiD degrader N-[5-(2-hydroxypropan-2-yl)-2-[(1r,4r-4-{[6-(2-{[2-(2,6-dioxopiperidin-3-yl)-1,3-dioxo-2,3-dihydro-1H-isoindol-4-yl]amino}ethyl)-2-azaspiro[3.3]heptan-2-yl]methyl}cyclohexyl]-1,3benzothiazol-6-yl]-6-(trifluoromethyl)pyridine-2-carboxamide (Compound A), and methods of use thereof.

### BACKGROUND OF THE INVENTION

IRAKIMiD degraders are subset of IRAK4 degraders with a unique profile that combines the activity of IRAK4 degradation and immunomodulatory imide drugs, or IMiDs, for the treatment of MYD88-mutant B-cell lymphomas. Oncogenic mutations of MYD88, most commonly MYD88L265P , are common in several subsets of B-cell lymphomas. In particular, MYD88 is estimated to be mutated in approximately 30-40% of ABC-DLBCL cases, 30-70% of primary CNS lymphoma cases, 45-75% of primary extranodal lymphomas cases, and more than 90% of Waldenström macroglobulinemia cases. The presence of MYD88 mutations is often associated with poorer response to chemotherapy and reduced overall survival compared to other genetic subtypes, supporting the need for more effective therapies targeting MYD88-mutated lymphoma.

Treatment of B-cell lymphomas typically involves front-line chemotherapy with a rituximab backbone. While effective in many other patients, front-line chemotherapy has significantly poorer survival rates in ABC-DLBCL. In additional lines of therapy, several novel targeted therapies have been approved recently, including polatuzumab, bendamustine, and chimeric antigen receptor T-cells. While these agents have some notable activity, many patients fail to respond to second line therapy or relapse from these therapies, with no adequate treatment options. Several targeted therapies that impact the NF-kB pathway, such as the Bruton's tyrosine kinase inhibitor ibrutinib, or the IMiD lenalidomide, have shown modest single agent activity, with poor durability of response in MYD88-mutated lymphomas.

In oncology, IRAK4 is an obligate protein in MYD88 signaling for which activated mutation is well characterized to drive oncogenesis and IMiDs are a class of drugs that degrade zinc-finger transcription factors, such as Ikaros and Aiolos, resulting in the restoration of Type 1 IFN signaling pathway which is also relevant in lymphoma. By combining the activity of the IMiDs with the IRAK4 degradation in a single agent addresses both the IL-1/TLR and the Type 1 IFN pathways synergistically while also demonstrating broad activity against MYD88-mutant B-cell lymphomas.

A need exists to develop formulations for IRAKIMiD degraders for use in cancer therapy.

### SUMMARY OF THE INVENTION

It has been found that IRAKIMiD degrader N-[5-(2-hydroxypropan-2-yl)-2-[(1r,4r-4-{[6-(2-{[2-(2,6-dioxopiperidin-3-yl)-1,3-dioxo-2,3-dihydro-1H-isoindol-4-yl]amino}ethyl)-2-azaspiro[3.3]heptan-2-yl]methyl}cyclohexyl]-1,3benzothiazol-6-yl]-6-(trifluoromethyl)pyridine -2-carboxamide (Compound A), and its salts, formulations and unit dosage forms, as described herein, have certain advantages in treating relapsed and/or refractory B-cell non-Hodgkin lymphomas.

Accordingly, in one aspect, the present disclosure provides a liquid formulation or unit dosage form comprising Compound A, or a pharmaceutically acceptable salt thereof, and a pharmaceutically acceptable excipient and/or carrier. In some embodiments, the liquid formulation or unit dosage form comprises Compound A at a concentration of about 0.05%-4.5% w/w of the total weight of the formulation or unit dosage form, respectively, or at a concentration of about 0.5-15 mg/mL.

In some embodiments, the liquid formulation or unit dosage form comprises a solubilizing agent (e.g., SBEBCD or HPBCD) at a concentration of about 10%-50% w/w of the total weight of the formulation or unit dosage form, respectively, or at a concentration of about 100-500 mg/mL. In some embodiments, the liquid formulation or unit dosage form comprises a pH modifier (e.g., hydrochloric acid or glacial acetic acid) at a concentration of about 0.5%-1.5% w/w of the total weight of the formulation or unit dosage form, respectively, or at a concentration of about 5-15 mg/mL. In some embodiments, the liquid formulation or unit dosage form is at about pH 2 to about pH 6. In some embodiments, the unit dosage form has a volume of from about 10 mL to about 50 mL.

In another aspect, the present invention provides a method for treating a relapsed and/or refractory B-cell non-Hodgkin lymphoma in a patient, comprising administering to the patient a therapeutically effective amount of Compound A, or a pharmaceutically acceptable salt thereof, or a liquid formulation described herein. In some embodiments, the relapsed and/or refractory B-cell non-Hodgkin lymphoma is selected from diffuse large B-cell lymphoma (DLBCL), active B-cell diffuse large B-cell lymphoma (ABC DLBCL), primary mediastinal B-cell lymphoma, primary extranodal lymphomas, primary CNS lymphoma, primary cutaneous large B-cell lymphoma, follicular lymphoma, chronic lymphocytic leukemia (CLL), small lymphocytic lymphoma (SLL), mantle cell lymphoma (MCL), marginal zone lymphomas, nodal marginal zone B-cell lymphoma, splenic marginal zone B-cell lymphoma, extranodal marginal zone B-cell lymphoma, mucosa-associated lymphoid tissue (MALT) lymphoma, Burkitt lymphoma, Waldenström macroglobulinemia, hairy cell leukemia (HCL), and primary intraocular lymphoma.

In some embodiments, the method comprises administering up to about 10.0 mg/kg of Compound A, or a pharmaceutically acceptable salt thereof, to the patient per day. In some embodiments, the method comprises administering Compound A, or a pharmaceutically acceptable salt thereof, to the patient intravenously. In some embodiments, the method comprises administering Compound A, or a pharmaceutically acceptable salt thereof, to the patient once every three weeks (Q3W), such as on day 1 of a 21-day cycle. In some embodiments, the method comprises administering Compound A, or a pharmaceutically acceptable salt thereof, to the patient twice every three weeks, such as on day 1 and 2 of a 21-day cycle.

These and other aspects of this disclosure will be apparent upon reference to the following detailed description. To this end, various references are set forth herein which describe in more detail certain background information and procedures and are each hereby incorporated by reference in their entirety.

### BRIEF DESCRIPTION OF THE DRAWINGS

FIG. 1 shows study schema of the dose escalation with MTD/RP2D confirmation (Phase 1a) and dose expansion (Phase 1b).

FIG. 2 depicts plasma concentration and PK in DL1 and DL2 showing a dose-proportional increase in exposure.

FIG. 3 shows the IRAK4, Ikaros, and Aiolos degradation in blood in DL1 and DL2 by PBMC FLOW.

FIG. 4 shows the IRAK4, Ikaros, and Aiolos degradation in tumor in DL1 by targeted MS.

### DETAILED DESCRIPTION OF CERTAIN EMBODIMENTS

### 1. General Description of Certain Embodiments of the Invention:

Compound A is a potent and selective, heterobifunctional small molecule therapeutic mediating the degradation of interleukin-1 receptor associated kinase 4 (IRAK4) and the immunomodulatory imide drug (IMiD) substrates Ikaros and Aiolos via the ubiquitin-proteasome system (UPS). The degradation of IRAK4 and IMiDs substrates is expected to maximize NF-κB inhibition while simultaneously upregulating the Type I Interferon response, thus restoring the apoptotic response and enabling oncogene-mediated cell death.

Accordingly, in some embodiments, the present disclosure provides a method for treating a relapsed and/or refractory B-cell non-Hodgkin lymphoma. In some embodiments, the present disclosure provides a method for treating a relapsed and/or refractory B-cell non-Hodgkin lymphoma in a patient, comprising administering to the patient a therapeutically effective amount of Compound A, or a pharmaceutically acceptable salt thereof, or a liquid formulation thereof as described herein.

In some embodiments, the present disclosure provides a method for treating diffuse large B-cell lymphoma (DLBCL) in a patient, comprising administering to the patient a therapeutically effective amount of Compound A, or a pharmaceutically acceptable salt thereof, or a liquid formulation thereof as described herein.

In some embodiments, the present disclosure provides a method for treating activated B-cell diffuse large B-cell lymphoma (ABC DLBCL) in a patient, comprising administering to the patient a therapeutically effective amount of Compound A, or a pharmaceutically acceptable salt thereof, or a liquid formulation thereof as described herein.

In some embodiments, the present disclosure provides a method for treating primary extranodal lymphomas in a patient, comprising administering to the patient a therapeutically effective amount of Compound A, or a pharmaceutically acceptable salt thereof, or a liquid formulation thereof as described herein.

In some embodiments, the present disclosure provides a liquid formulation, which comprises Compound A, or a pharmaceutically acceptable salt thereof, and a pharmaceutically acceptable excipient and/or carrier. In some embodiments, the present disclosure provides a unit dosage form, which comprises Compound A, or a pharmaceutically acceptable salt thereof, and a pharmaceutically acceptable excipient and/or carrier.

In the following disclosure, certain specific details are set forth in order to provide a thorough understanding of various embodiments. However, one skilled in the art will understand that the methods and uses described herein may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment.

Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

### 2. Definitions

As used in the specification and appended claims, unless specified to the contrary, the following terms and abbreviations have the meaning indicated:

As used herein, the terms “about” or “approximately” have the meaning of within 20% of a given value or range. In some embodiments, the term “about” refers to within 20%, 19%, 18%, 17%, 16%, 15%, 14%, 13%, 12%, 11%, 10%, 9%, 8%, 7%, 6%, 5%, 4%, 3%, 2%, or 1% of a given value.

As used herein, “Compound A” refers to IRAKIMID degrader N-[5-(2-hydroxypropan-2-yl)-2-[(1r,4r-4-{[6-(2-{[2-(2,6-dioxopiperidin-3-yl)-1,3-dioxo-2,3-dihydro-1H-isoindol-4-yl]amino}ethyl)-2-azaspiro[3.3]heptan-2-yl]methyl}cyclohexyl]-1,3benzothiazol-6-yl]-6-(trifluoromethyl)pyridine-2-carboxamide, of formula:

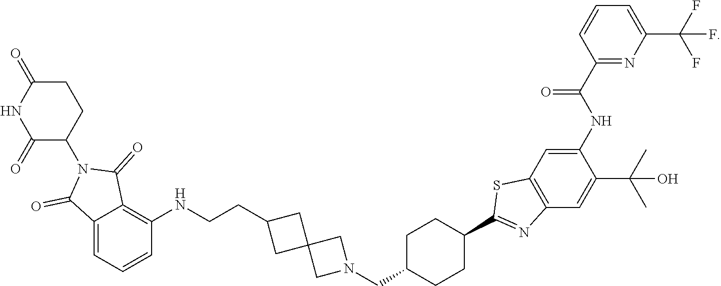 [SMILES: CC(C)(O)c1cc2nc([C@H]3CC[C@H](CN4CC5(CC(CCNc6cccc7c6C(=O)N(C6CCC(=O)NC6=O)C7=O)C5)C4)CC3)sc2cc1NC(=O)c1cccc(C(F)(F)F)n1]

In some embodiments, Compound A is provided in solid form. In some embodiments, Compound A is amorphous.

As used herein, “Compound (R)-A” refers to IRAKIMID degrader N-[5-(2-hydroxypropan-2-yl)-2-[(1r,4r-4-{[6-(2-{[2-((R)-2,6-dioxopiperidin-3-yl)-1,3-dioxo-2,3-dihydro-1H-isoindol-4-yl]amino}ethyl)-2-azaspiro[3.3]heptan-2-yl]methyl}cyclohexyl]-1,3benzothiazol-6-yl]-6-(trifluoromethyl)pyridine-2-carboxamide, of formula:

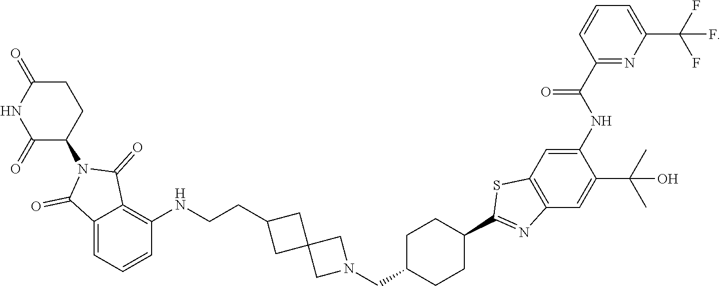 [SMILES: CC(C)(O)c1cc2nc([C@H]3CC[C@H](CN4CC5(CC(CCNc6cccc7c6C(=O)N([C@@H]6CCC(=O)NC6=O)C7=O)C5)C4)CC3)sc2cc1NC(=O)c1cccc(C(F)(F)F)n1]

In some embodiments, Compound (R)-A is provided in solid form. In some embodiments, Compound (R)-A is amorphous.

As used herein, “Compound (S)-A” refers to IRAKIMID degrader N-[5-(2-hydroxypropan-2-yl)-2-[(1r,4r)-4-{[6-(2-{[2-((S)-2,6-dioxopiperidin-3-yl)-1,3-dioxo-2,3-dihydro-1H-isoindol-4-yl]amino}ethyl)-2-azaspiro[3.3]heptan-2-yl]methyl}cyclohexyl]-1,3benzothiazol-6-yl]-6-(trifluoromethyl)pyridine-2-carboxamide, of formula:

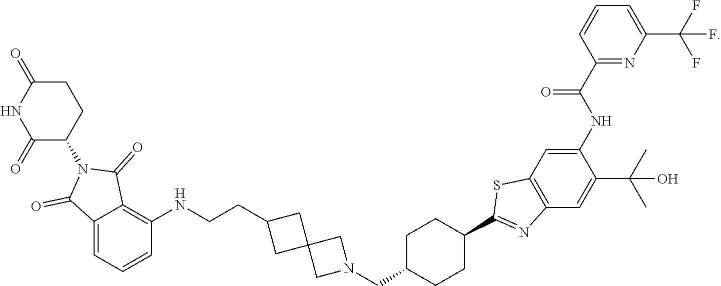 [SMILES: CC(C)(O)c1cc2nc([C@H]3CC[C@H](CN4CC5(CC(CCNc6cccc7c6C(=O)N([C@H]6CCC(=O)NC6=O)C7=O)C5)C4)CC3)sc2cc1NC(=O)c1cccc(C(F)(F)F)n1]

In some embodiments, Compound (S)-A is provided in solid form. In some embodiments, Compound (S)-A is amorphous.

As used herein, the term “IRAKIMID degrader” refers to an agent that degrades IRAK4 and other IMiD targets. Various IRAKIMID degraders have been described previously, for example, in WO 2019/133531, WO 2020/010227, and WO 2021/127190, the contents of each of which are incorporated herein by reference in their entireties. In certain embodiments, an IRAKIMiD degrader has an DC₅₀ of less than about 50 μM, less than about 1 μM, less than about 500 nM, less than about 100 nM, less than about 10 nM, or less than about 1 nM.

As used herein, the term “mg/kg” or “mpk” refers to the milligram of medication (for example, Compound A) per kilogram of the body weight of the subject taking the medication.

As used herein, the term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium, and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate.

The term “patient,” as used herein, means an animal, preferably a mammal, and most preferably a human. In some embodiments, the patient is a treatment-naïve patient.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, a patient or subject “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment or therapy.

A “therapeutically effective amount” or “therapeutically effective dosage” of a drug or therapeutic agent, such as Compound A, or a pharmaceutically acceptable salt thereof, is any amount of the drug that, when used alone or in combination with another therapeutic agent, protects a patient or subject against the onset of a disease, such as LGL-L, or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

In preferred embodiments, a therapeutically effective amount of the drug, such as Compound A, promotes regression to the point of eliminating the disease. In addition, the terms “effective” and “effectiveness” with regard to a treatment includes both pharmacological effectiveness and physiological safety. Pharmacological effectiveness refers to the ability of the Compound A, or a pharmaceutically acceptable salt thereof, to treat the disease in the patient. Physiological safety refers to the level of toxicity, or other adverse physiological effects at the cellular, organ and/or organism level (adverse effects) resulting from administration of the drug.

As used herein, the terms “therapeutic benefit” or “benefit from therapy” refers to an improvement in one or more of overall survival, progression-free survival, partial response, complete response, and overall response rate and can also include a decrease in severity of disease symptoms, an increase in frequency and duration of disease symptom-free periods, or a prevention of impairment or disability due to the disease affliction.

The phase “woman of childbearing potential” (WOCBP) are considered fertile: 1. Following menarche; 2. From the time of menarche until becoming postmenopausal unless permanently sterile. A postmenopausal state is defined as no menses for 12 months without an alternative medical cause. A high follicle-stimulating hormone (FSH) level in the postmenopausal range may be used to confirm a postmenopausal state in women not using hormonal contraception or hormonal replacement therapy (HRT). However, in the absence of 12 months of amenorrhea, confirmation with more than one FSH measurement is required. Females on HRT and whose menopausal status is in doubt will be required to use one of the non-estrogen hormonal highly effective contraception methods if they wish to continue their HRT during the study. Otherwise, they must discontinue HRT to allow confirmation of postmenopausal status before study enrollment. Permanent sterilization methods (for the purpose of this study) include: documented hysterectomy; documented bilateral salpingectomy' documented bilateral oophorectomy; for individuals with permanent infertility due to an alternate medical cause other than the above, (e.g., Mullerian agenesis, androgen insensitivity, gonadal dysgenesis), Investigator discretion should be applied to determining study entry.

### 3. Description of Exemplary Embodiments

The IRAKIMiD degraders provided herein are heterobifunctional small molecule therapeutic targeting CRBN E3 ligase and IRAK4 to mediate the selective degradation of IRAK4 protein as well as IMiD targets, including Ikaros and Aiolos. In MYD88-mutant B-cell lymphoma, degradation of the Myddosome component IRAK4, in combination with IMiD-mediated degradation of Ikaros and Aiolos and the resulting downregulation of IRF4 and activation of an interferon-like response, will synergize to induce cell death and antitumor responses. In certain embodiments, provided herein is a treatment of adult patients with MYD88-mutant B-cell lymphoma who have received at least one prior therapy. The IRAKIMiD degraders of the current invention are provided by intravenous administration at the doses and schedules described herein.

In some embodiments, the present disclosure provides a method for treating a relapsed and/or refractory B-cell non-Hodgkin lymphoma in a patient, comprising administering to the patient a therapeutically effective amount of Compound A, or a pharmaceutically acceptable salt thereof, or a liquid formulation thereof as described herein.

In some embodiments, the present disclosure provides a method for treating diffuse large B-cell lymphoma (DLBCL) in a patient, comprising administering to the patient a therapeutically effective amount of Compound A, or a pharmaceutically acceptable salt thereof, or a liquid formulation thereof as described herein.

In some embodiments, the present disclosure provides a method for treating activated B-cell diffuse large B-cell lymphoma (ABC DLBCL) in a patient, comprising administering to the patient a therapeutically effective amount of Compound A, or a pharmaceutically acceptable salt thereof, or a liquid formulation thereof as described herein.

In some embodiments, the present disclosure provides a method for treating primary extranodal lymphomas in a patient, comprising administering to the patient a therapeutically effective amount of Compound A, or a pharmaceutically acceptable salt thereof, or a liquid formulation thereof as described herein.

In some embodiments, the patient is male or female aged ≥18 years.

In some embodiments, the patient has histologically confirmed diagnosis of B-cell NHL according to the World Health Organization (WHO) classification. In some embodiments, a patient has evidence of histological transformation of indolent lymphoma to DLBCL.

In some embodiments, a method comprises analyzing fresh/archival formalin fixed paraffin embedded (FFPE) tumor tissue of a patient preferably collected within 6 months prior to first dose (C1D1). In some embodiments, a method comprises analyzing a blood sample of a patient for central testing of MYD88 mutational analysis.

In some embodiments, the patient has histologically confirmed diagnosis of DLBCL according to the WHO classification. In some embodiments, the patient has evidence of histological transformation to DLBCL from an earlier diagnosis of low-grade lymphoma with subsequent DLBCL relapse.

In some embodiments, the patient has documented tumor MYD88 status (as mutant or wild type).

In some embodiments, the patient has disease relapsed and/or refractory to at least 1 accepted standard systemic regimen.

In some embodiments, the patient has at least one bi-dimensionally measurable disease site. In some embodiments, the patient has a lesion with a greatest transverse diameter of at least 1.5 cm and greatest perpendicular diameter of at least 1.0 cm. In some embodiments, the patient has a lesion which is positive on positron emission tomography (PET) scan.

In some embodiments, the patient has Eastern Cooperative Oncology Group (ECOG) performance status of 0-2.

In some embodiments, the patient has an adequate organ and hematologic function on C1D1 (pre-dose), which is defined as one or more of the following:
Hematology
   absolute neutrophil count (ANC)≥1000/μL;
   hemoglobin≥8.0 g/dL (for those patients undergoing red blood cell [RBC] transfusion, hemoglobin must be evaluated at least 14 days after the last RBC transfusion);
   platelet count≥75,000/μL (for those patients undergoing transfusion, platelet count must be evaluated at least 7 days after the last platelet transfusion);
Hepatic Function
   aspartate aminotransferase (AST), alanine transaminase (ALT)≤3× upper limit of normal (ULN) or ≤5× ULN in cases of documented lymphoma involvement of liver;
   total serum bilirubin≤1.5× ULN or ≤5× ULN if secondary to Gilbert's syndrome or documented lymphoma involvement of liver;
Renal Function
   serum electrolyte (potassium, calcium, and magnesium) levels within the normal reference range (may be supplemented according to institutional standard); and
   serum creatinine clearance≥60 mL/min/1.73 m² either measured or calculated using standard Cockcroft-Gault formula.

In some embodiments, the patient has a negative SARS-CoV-2 test.

In some embodiments, the patient is a woman of childbearing potential (WOCBP) and uses two highly effective contraceptive methods for the duration of the treatment with compound A as described herein and 6 months after the last dose of the treatment with compound A as described herein.

In some embodiments, the WOCBP patient has a negative serum pregnancy test, for example, within 72 hours prior to the first dose of the treatment with compound A as described herein.

In some embodiments, the patient is male and uses two highly effective contraceptive methods during the treatment with compound A as described herein and for 6 months after the last dose of the treatment with compound A as described herein if the partner is a WOCBP.

In some embodiments, the patient has no known central nervous system (CNS) lymphoma or meningeal involvement.

In some embodiments, the patient has no history of malignancy other than B-cell NHL or DLBCL unless the patient has been disease-free for ≥2 years. Exceptions to the ≥2-year time limit include treated basal cell or localized squamous cell skin carcinoma, localized prostate cancer, or other localized carcinomas such as carcinoma in situ of cervix, breast, or bladder.

In some embodiments, the patient has no active concurrent malignancy with the exception of basal cell or localized squamous cell skin carcinoma, localized prostate cancer, or other localized carcinomas such as carcinoma in situ of cervix, breast, or bladder.

In some embodiments, the patient is not a patient who has not recovered from any clinically significant adverse events (AEs) of previous treatments to pre-treatment baseline or Grade 1 prior to first dose of the treatment with compound A as described herein.

In some embodiments, the patient has no ongoing unstable cardiovascular function: symptomatic ischemia, or uncontrolled clinically significant conduction abnormalities (i.e., ventricular tachycardia on anti-arrhythmia are excluded; 1st degree atrioventricular block or asymptomatic left anterior fascicular block/right bundle branch block will not be excluded), or congestive heart failure of New York Heart Association Class≥III, or myocardial infarction.

In some embodiments, the patient has no congenital long QT syndrome, or a QT interval corrected by Fridericia's formula (QTcF)≥450 ms (average of triplicate ECGs) on C1D1 (pre-dose) with the exception of a documented bundle branch block or unless secondary to pacemaker.

In some embodiments, the patient has no thromboembolic or cerebrovascular event (i.e., transient ischemic attacks, cerebrovascular accidents, pulmonary emboli, or clinically significant deep vein thrombosis)≤6 months prior to first dose of the treatment with compound A as described herein.

In some embodiments, the patient has no infection requiring antibiotics, antivirals, or antifungals within 1 week prior to first dose of the treatment with compound A as described herein, unless such infection is adequately controlled (defined as exhibiting no ongoing signs/symptoms related to the infection and with clinical improvement).

In some embodiments, the patient has no positive hepatitis B and/or hepatitis C serology or known seropositivity for or history of active viral infection with human immunodeficiency virus (HIV).

In some embodiments, the patient has no concurrent medical conditions including psychiatric disorders.

In some embodiments, the patient is not pregnant or breast feeding

In some embodiments, the patient has no prior treatment with an IRAK4 inhibitor.

In some embodiments, the patient has no disease progression on IMiD-containing regimen <6 months prior to first dose of the treatment with compound A as described herein.

In some embodiments, the patient has no relapsed/refractory disease after ≥2 prior IMiD-containing regimens.

In some embodiments, the patient has no discontinuation of prior IMiD therapy due to IMiD-related toxicity.

In some embodiments, the patient has no prior allogeneic hematopoietic stem cell transplant.

In some embodiments, the patient has no autologous hematopoietic stem cell transplant within 6 months prior to first dose of the treatment with compound A as described herein. In some embodiments, the patient has not progressed within 6 months from the day of stem cell infusion.

In some embodiments, the patient has no radiation treatment within 4 weeks prior to first dose of the treatment with compound A as described herein, unless the tumor site continues to increase in size after the patient has completed radiotherapy treatment.

In some embodiments, the patient has no major surgery requiring general anesthesia within 4 weeks prior to first dose of the treatment with compound A as described herein.

In some embodiments, the patient has not received live vaccine within 1 month prior to the first dose of the treatment with compound A as described herein.

In some embodiments, the patient has no exposure to investigational or non-investigational anti-cancer therapy within 2 weeks or within at least 4 half-lives (up to a maximum of 4 weeks) prior to the first dose of the treatment with compound A as described herein, whichever is longer. In all situations, the maximum washout period will not exceed 4 weeks prior to first dose of the treatment with compound A as described herein.

In some embodiments, the patient has not completed a course of SARS-CoV-2 vaccine within 14 days prior to first dose of the treatment with compound A as described herein.

In some embodiments, a method of the present invention comprises intravenously administering a liquid formulation as described herein. In some embodiments, a method of the present invention comprises administering a unit dosage form as described herein. In some embodiments, a method of the present invention comprises administering daily to a patient a liquid formulation or a unit dosage form as described herein.

Liquid Formulations

According to one embodiment, the invention provides a liquid formulation comprising a IRAKIMiD degrader of this invention (e.g., Compound A) or a pharmaceutically acceptable derivative thereof and a pharmaceutically acceptable excipient (e.g., a solubilizing agent and a pH modifier) and/or carrier (e.g., water). The amount of Compound A in liquid formulations of this invention is such that it is effective to measurably degrade and/or inhibit IRAK4 protein as well as IMiD targets, including Ikaros and Aiolos, or mutants thereof, in a patient. The liquid formulation of the present invention may be administered parenterally by injection, infusion, or implantation (intravenous, intramuscular, subcutaneous, or the like) as the liquid formulation or in unit dosage forms or via suitable delivery devices or implants containing conventional, non-toxic pharmaceutically acceptable excipients and carriers. In certain embodiments, a liquid formulation of this invention is formulated for administration to a patient in need of such composition. In some embodiments, a composition of this invention is formulated for parenteral (e.g., intravenous) administration to a patient.

The liquid formulations comprising an IRAKIMiD degrader of this invention (e.g., Compound A) can be prepared in water or another solvent or dispersing medium suitably mixed with one or more pharmaceutically acceptable excipients including, but not limited to surfactants, dispersants, emulsifiers, viscosity modifying agents, solubilizing agents, pH modifiers, and combinations thereof.

In some embodiments, a provided liquid formulation for parenteral use is provided in unit dosage forms (e.g., in single-dose ampoules), or in vials containing several doses and in which a suitable preservative may be added (see below). Typically, such compositions can be prepared as injectable formulations, for example, solutions or suspensions; solid and liquid forms suitable for using to prepare solutions or suspensions upon the addition of a reconstitution or dilution medium prior to injection. In preferred embodiments, the liquid formulation or unit dosage forms thereof are administered intravenously.

In some embodiments, a liquid formulation or unit dosage form of the invention comprises Compound A, or a pharmaceutically acceptable salt thereof, at a concentration of about 0.05%-1.5% w/w of the total weight of the formulation or unit dosage form. In some embodiments, a liquid formulation or unit dosage form of the invention comprises Compound A, or a pharmaceutically acceptable salt thereof, at a concentration of about 0.05%-0.5%, about 0.1%-1.0%, about 0.6%-1.4%, about 0.7%-1.3%, about 0.8%-1.2%, or about 0.9%4.1% w/w of the total weight of the formulation or unit dosage form. In some embodiments, a liquid formulation or unit dosage form of the invention comprises Compound A, or a pharmaceutically acceptable salt thereof, at a concentration of about 0.60%, about 0.65%, about 0.70%, about 0.75%, about 0.80%, about 0.85%, about 0.90%, about 0.95%, about 1.00%, about 1.05%, about 1.10%, about 1.15%, about 1.20%, about 1.25%, about 1.30%, about 1.35%, about 1.40%, about 1.45%, or about 1.50% w/w of the total weight of the formulation or unit dosage form.

In some embodiments, a liquid formulation or unit dosage form of the invention comprises Compound A, or a pharmaceutically acceptable salt thereof, at a concentration of about 0.5-15 mg/mL. In some embodiments, a liquid formulation or unit dosage form of the invention comprises Compound A, or a pharmaceutically acceptable salt thereof, at a concentration of about 0.5-5 mg/mL, about 1-10 mg/mL, about 6-14 mg/mL, about 6.5-13.5 mg/mL, about 7-13 mg/mL, about 7.5-12.5 mg/mL, about 8-12 mg/mL, about 8.5-11.5 mg/mL, about 9-11 mg/mL, or about 9.5-10.5 mg/mL. In some embodiments, a liquid formulation or unit dosage form of the invention comprises Compound A, or a pharmaceutically acceptable salt thereof, at a concentration of about 8 mg/mL, about 8.5 mg/mL, about 9 mg/mL, about 9.5 mg/mL, about 10 mg/mL, about 10.5 mg/mL, about 11 mg/mL, about 11.5 mg/mL, or about 12 mg/mL.

In some embodiments, the liquid formulation or unit dosage form of the invention includes a solubilizing agent. In some embodiments, the solubilizing agent is a cyclodextrin. Cyclodextrines include members of a family of cyclic oligosaccharides, composed of 5 or more a-D-glucopyranoside units linked between positions 1 and 4, as known for amylose, a fragment of starch. In some embodiments, the cyclodextrin is an alpha-cyclodextrin, beta-cyclodextrin, and/or gamma-cyclodextrin. In some embodiments, the cyclodextrin is a cyclodextrin disclosed in November 2014 EMA/CHMP/333892/2013 Committee for Human Medicinal Products (CHMP), the entire contents of which are herein incorporated by reference. In certain embodiments, the cyclodextrin is a beta-cyclodextrin, such as sulfobutylether-beta-cyclodextrin (SBEBCD) or hydroxypropyl-beta-cyclodextrin (HPBCD). In some embodiments, the incorporation of a beta-cyclodextrin (e.g., SBEBCD or HPBCD) in the intravenous compositions of the present invention improve the dissolution of Compound A, or a pharmaceutically acceptable salt thereof, to provide a clear homogeneous solution for injection. In some embodiments, a liquid formulation or unit dosage form of the invention comprises a solubilizing agent (e.g., SBEBCD or HPBCD) at a concentration of about 10%-50% w/w of the total weight of the formulation or unit dosage form. In some embodiments, a liquid formulation or unit dosage form of the invention comprises a solubilizing agent (e.g., SBEBCD or HPBCD) at a concentration of about 15%-25%, about 20%-30%, or about 25%-35% w/w of the total weight of the formulation or unit dosage form. In some embodiments, a liquid formulation or unit dosage form of the invention comprises a solubilizing agent (e.g., SBEBCD or HPBCD) at a concentration of about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, or about 35% w/w of the total weight of the formulation or unit dosage form.

In some embodiments, a liquid formulation or unit dosage form of the invention comprises a solubilizing agent (e.g., SBEBCD or HPBCD) at a concentration of about 100-500 mg/mL of the total weight of the formulation or unit dosage form. In some embodiments, a liquid formulation or unit dosage form of the invention comprises a solubilizing agent (e.g., SBEBCD or HPBCD) at a concentration of about 100-300, about 200-400, or about 300-500 mg/mL. In some embodiments, a liquid formulation or unit dosage form of the invention comprises a solubilizing agent (e.g., SBEBCD or HPBCD) at a concentration of about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 370, about 380, about 390, about 400, about 410, about 420, about 430, about 440, about 450, about 460, about 470, about 480, or about 490 mg/mL.

In some embodiments, the liquid formulation or unit dosage form of the invention includes a pH modifier. Suitable pH modifiers in include acidity regulators (e.g., acid or bases) or buffers (e.g., acetate, citrate, phosphate, histidine, etc.). In some embodiments, the liquid formulation or unit dosage form of the invention includes an acidity regulator. In some embodiments, the acidity regulator is an inorganic acid (e.g., hydrochloric acid, phosphoric acid, etc.) or an organic acid (e.g., acetic acid, oxalic acid, maleic acid, tartaric acid, citric acid, succinic acid, malonic acid, etc.). In some embodiment, the acidity regulator in an inorganic acid, such as hydrochloric acid. In certain embodiments, the acidity regulator is an organic acid, such as glacial acetic acid. In some embodiments, the incorporation of an acidity regulator in the liquid formulation or unit dosage form of the invention lowers the pH to increase the solubility of Compound A in the liquid formulation or unit dosage form (e.g., reduces precipitation of Compound A). In some embodiments, a liquid formulation or unit dosage form of the invention comprises an acidity regulator (e.g., hydrochloric acid or glacial acetic acid) at a concentration of about 0.5%-1.5% w/w of the total weight of the formulation or unit dosage form. In some embodiments, a liquid formulation of the invention comprises an acidity regulator (e.g., hydrochloric acid or glacial acetic acid) at a concentration of about 0.6%-1.4%, about 0.7%-1.3%, about 0.8%-1.2%, or about 0.9%-1.1% w/w of the total weight of the formulation or unit dosage form. In some embodiments, a liquid formulation of the invention comprises an acidity regulator (e.g., hydrochloric acid or glacial acetic acid) at a concentration of about 0.60%, about 0.65%, about 0.70%, about 0.75%, about 0.80%, about 0.85%, about 0.90%, about 0.95%, about 1.00%, about 1.05%, about 1.10%, about 1.15%, about 1.20%, about 1.25%, about 1.30%, about 1.35%, about 1.40%, about 1.45%, or about 1.50% w/w of the total weight of the formulation or unit dosage form.

In some embodiments, a liquid formulation or unit dosage form of the invention comprises an acidity regulator (e.g., hydrochloric acid or glacial acetic acid) at a concentration of about 5-15 mg/mL. In some embodiments, a liquid formulation of the invention comprises an acidity regulator (e.g., hydrochloric acid or glacial acetic acid) at a concentration of about 6-14 mg/mL, about 7-13 mg/mL, about 8-12 mg/mL, or about 9-11 mg/mL. In some embodiments, a liquid formulation of the invention comprises an acidity regulator (e.g., hydrochloric acid or glacial acetic acid) at a concentration of about 6.5 mg/mL, about 7.0 mg/mL, about 7.5 mg/mL, about 8.0 mg/mL, about 8.5 mg/mL, about 9.0 mg/mL, about 9.5 mg/mL, about 10.0 mg/mL, about 10.5 mg/mL, about 11.0 mg/mL, about 11.5 mg/mL, about 12.0 mg/mL, about 12.5 mg/mL, about 13.0 mg/mL, about 13.5 mg/mL, about 14.0 mg/mL, or about 14.5 mg/mL.

In some embodiments, a liquid formulation or unit dosage form of the invention comprises a pH of from about pH 2 to about pH 6. In some embodiments, the pH of a liquid formulation or unit dosage form of the invention is from about pH 3 to about pH 5. In some embodiments, the pH of a liquid formulation or unit dosage form of the invention is about pH 3.1, about pH 3.2, about pH 3.3, about pH 3.4, about pH 3.5, about pH 3.6, about pH 3.7, about pH 3.8, about pH 3.9, about pH 4.0, about pH 4.1, about pH 4.2, about pH 4.3, about pH 4.4, about pH 4.5, about pH 4.6, about pH 4.7, about pH 4.8, or about pH 4.9.

In some embodiments, a liquid formulation or unit dosage form of the invention comprises a carrier or dispersion medium containing, for example, water, surfactants, co-solvents, such as ethanol or one or more polyols (e.g., glycerol, propylene glycol, and liquid polyethylene glycol), oils, such as vegetable oils (e.g., peanut oil, corn oil, sesame oil, etc.), or combinations thereof. In some embodiments, the surfactant is selected from a) natural and synthetic lipophilic agents including phospholipids, cholesterol, and cholesterol fatty acid esters and derivatives thereof; b) nonionic surfactant including polyoxyethylene fatty alcohol esters, sorbitan fatty acid esters (Spans), polyoxyethylene sorbitan fatty acid esters [e.g., polyoxyethylene (20) sorbitan monooleate (Tween 80), polyoxyethylene (20) sorbitan monostearate (Tween 60), polyoxyethylene (20) sorbitan monolaurate (Tween 20) and other Tweens], sorbitan esters, glycerol esters [e.g., Myrj and glycerol triacetate (triacetin)], polyethylene glycols [e.g., tocopherol polyethylene glycol succinate (TPGS)], cetyl alcohol, cetostearyl alcohol, stearyl alcohol, polysorbate 80, poloxamers, poloxamines, polyoxyethylene castor oil derivatives (e.g., Cremophor® RH40, Cremphor A25, Cremphor A20, Cremophor® EL, and other Cremophors), sulfosuccinates, alkyl sulphates (SLS), PEG glyceryl fatty acid esters [e.g., PEG-8 glyceryl caprylate/caprate (Labrasol), PEG-4 glyceryl caprylate/caprate (Labrafac Hydro WL 1219), PEG-32 glyceryl laurate (Gelucire 444/14), PEG-6 glyceryl mono oleate (Labrafil M 1944 CS), PEG-6 glyceryl linoleate (Labrafil M 2125 CS)], propylene glycol mono- and di-fatty acid esters (e.g., propylene glycol laurate, propylene glycol caprylate/caprate, Brij® 700, ascorbyl-6-palmitate, stearylamine, sodium lauryl sulfate, polyoxethyleneglycerol triiricinoleate), polyethylene glycol or any combinations or mixtures thereof; c) anionic surfactants including calcium carboxymethylcellulose, sodium carboxymethylcellulose, sodium sulfosuccinate, dioctyl, sodium alginate, alkyl polyoxyethylene sulfates, sodium lauryl sulfate, triethanolamine stearate, potassium laurate, bile salts, or any combinations or mixtures thereof; and d) cationic surfactants (e.g., cetyltrimethylammonium bromide and lauryldimethylbenzylammonium chloride), and combinations thereof. In preferred aspects, water (e.g., water for injection or WFI) is added to the formulation or unit dosage form of the present invention.

In some embodiments, the present invention provides a unit dosage form, which is a liquid formulation of the present invention, as described above, with a volume of from about 10 mL to about 50 mL. In some embodiments, the present invention provides a unit dosage form, which is a liquid formulation of the present invention, as described above, with a volume of about 10 mL, about 15 mL, about 20 mL, about 25 mL, about 30 mL, about 35 mL, about 40 mL, about 45 mL, or about 50 mL.

In some embodiments, a liquid formulation or unit dosage form of the invention comprises Compound A or a pharmaceutically acceptable salt thereof, about 5-15% w/w (e.g., about 10% w/w) of solubilizing agent (e.g., HPBCD), optionally about 1-10% w/w (e.g., about 5% w/w) of nonionic surfactant (e.g., TPGS), optionally about 0.05-0.2M acetate (e.g., about 0.1M acetate), and water to about 1-20 mg/mL (e.g., about 1, 2.5, 5, 10 or 20 mg/mL) concentration of Compound A.

Sterile injectable solutions can be prepared by incorporating the active compounds in the required amount in the appropriate solvent or dispersion medium with one or more of the excipients listed above, as required, followed by filtered sterilization. Generally, dispersions are prepared by incorporating the various sterilized active ingredients into a sterile vehicle which contains the basic dispersion medium and the required other ingredients from those listed above.

In some embodiments, a liquid formulation or unit dosage form of the invention is filtered through one or more, preferably two, sterilizing filters with 0.22 μm pore size attached optionally in series to obtain a sterilized solution of Compound A. In some embodiments, the sterile solution is then filled into glass vials, stoppered, and sealed aseptically.

In some embodiments, a liquid formulation or unit dosage form of the present invention is a stabilized liquid formulation or stabilized unit dosage form. In some embodiments, the liquid formulation or unit dosage form is stable (e.g., maintains greater than 99% purity of Compound A, or a pharmaceutically acceptable salt thereof, in the formulation or unit dosage form) for at least about 1 hour, about 2 hours, about 3 hours, about 4 hours, about 6 hours, 12 hours, about 18 hours, about 24 hours, about 36 hours, or about 48 hours under ambient laboratory lighting and temperature (e.g., 15 to 25° C.). In some embodiments, a liquid formulation or unit dosage form of the present invention is in frozen form (e.g., about −20° C.). In some embodiments, a liquid formulation or unit dosage form of the present invention is stable for at least about 1 week, about 2 weeks, about 3 weeks, about 4 weeks, about 5 weeks, about 6 weeks, about 7 weeks, about 8 weeks, about 9 weeks, about 10 weeks, about 11 weeks, or at least about 12 weeks in frozen form (e.g., about −20° C.). In some embodiments, a liquid formulation or unit dosage form of the present invention is stable for at least about 1 month, about 2 months, about 3 months, about 4 months, about 5 months, about 6 months, about 7 months, about 8 months, about 9 months, about 10 months, about 11 months, or at least about 12 months in frozen form (e.g., about −20° C.). In some embodiments, a liquid formulation or unit dosage form of the present invention is stable for at least 2 months at about −20° C. In some embodiments, a liquid formulation or unit dosage form of the present invention is stable after 3 freeze/thaw cycles.

In some embodiments, a liquid formulation or unit dose form of the present invention is mixed with an IV infusion vehicle before IV administration. In some embodiments, a liquid formulation or unit dose form of the present invention is mixed with an IV infusion vehicle using a transfer kit (e.g., close system transfer device or CSTD). In some embodiments, the IV infusion vehicle is an injectable medium such as 5% dextrose (D5W). In some embodiments, a liquid formulation or unit dose form of the present invention is diluted into a 5% dextrose IV bag (e.g., 500 mL) for IV administration. In some embodiments, the present invention provides an IV bag comprising a unit dose form of the present invention and an IV infusion vehicle (e.g., 5% dextrose) for IV administration. In some embodiments, an IV infusion vehicle comprises a volume of from about 100 mL to about 1000 mL, preferably about 500 mL (e.g., of 5% dextrose). In some embodiments, an IV bag comprising a unit dose form of the present invention and an IV infusion vehicle (e.g., 5% dextrose) is stable (e.g., maintains 95.0 to 100.0% purity of Compound A, or a pharmaceutically acceptable salt thereof, in the IV bag) for at least about 1 hour, about 2 hours, about 3 hours, about 4 hours, about 6 hours, 12 hours, about 18 hours, about 24 hours, about 36 hours, or about 48 hours under ambient laboratory lighting and temperature (e.g., 15 to 25° C.). In some embodiments, the IV bag stored under ambient laboratory lighting and temperature (e.g., 15 to 25° C.) is stable for at least about 48 hours (e.g., 24-48 hours) before IV administration.

Dosing and Schedules

As provided in view of preclinical data described herein, an IKAKIMiD degrader (e.g., Compound A) or a pharmaceutically acceptable salt thereof, or a pharmaceutical composition thereof, is administered to a patient at a dose and schedule appropriate to give the desired cancer regression effect with minimum side effects. In some embodiments, the dose of IKAKIMiD degrader (e.g., Compound A) or a pharmaceutically acceptable salt thereof, or a pharmaceutical composition thereof, is appropriate to achieve tumor regression and substantial IRAK4 and Ikaros degradation. In some embodiments, a method of the present invention comprises administering to a patient at a dosage of about 0.1 mg/kg to about 10 mg/kg of Compound A, such as about 0.1 mg/kg to about 5 mg/kg, about 0.5 mg/kg to about 5 mg/kg, about 1 mg/kg to about 5 mg/kg, about 1 mg/kg to about 7 mg/kg, about 3 mg/kg to about 8 mg/kg, about 5 mg/kg to about 8 mg/kg, or about 5 mg/kg to about 10 mg/kg of Compound A. In some embodiments, a method of the present invention comprises administering to a patient at a dosage of up to about 0.1 mg/kg, up to about 1 mg/kg, up to about 3 mg/kg, up to about 5 mg/kg, or up to about 10.0 mg/kg of Compound A, for example, at a dosage of about 0.1 mg/kg, about 0.2 mg/kg, about 0.3 mg/kg, about 0.4 mg/kg, about 0.5 mg/kg, about 1.0 mg/kg, 1.5 mg/kg , about 2.0 mg/kg, about 2.5 mg/kg , about 3.0 mg/kg , about 3.5 mg/kg , about 4.0 mg/kg, about 4.5 mg/kg, about 5.0 mg/kg, about 5.5 mg/kg, about 6.0 mg/kg, about 6.5 mg/kg, about 7.0 mg/kg, about 7.5 mg/kg, about 8.0 mg/kg, about 8.5 mg/kg, about 9.0 mg/kg, or about 9.5 mg/kg of Compound A. In some embodiments, a method of the present invention comprises administering to a patient about 0.16 mg/kg, about 0.32 mg/kg, about 0.64 mg/kg, about 1.25 mg/kg, about 2.0 mg/kg, about 3.0 mg/kg, about 4.2 mg/kg, or about 5.6 mg/kg of Compound A on day 1 of a 21-day cycle. In some embodiments, a method of the present invention comprises administering to a patient about 0.16 mg/kg, about 0.32 mg/kg, about 0.64 mg/kg, about 1.25 mg/kg, about 2.0 mg/kg, about 3.0 mg/kg, about 4.2 mg/kg, or about 5.6 mg/kg of Compound A on day 1 and day 2 of a 21-day cycle. In certain embodiments, the amount and schedule of Compound A administered to a patient is provided in Example 2 below.

In some embodiments, a method of the present invention comprises administering a liquid formulation or a unit dosage form as described herein, wherein there is at least 24 hours between two consecutive administrations. In some embodiments, a method of the present invention comprises administering a liquid formulation or a unit dosage form as described herein, wherein there are about 1-7 days between two consecutive administrations. In some embodiments, there are about 1, about 2, about 3, about 4, about 5, about 6, or about 7 days between two consecutive administrations. In certain embodiments, a liquid formulation or a unit dosage form as described herein is administered every 7 days between two consecutive administrations.

In some embodiments, a method of the present invention comprises administering a liquid formulation or a unit dosage form as described herein, wherein there is about 1-4 weeks between two consecutive administrations. In some embodiments, there is about 1, about 2, about 3, or about 4 weeks between two consecutive administrations. In some embodiments, a liquid formulation or a unit dosage form as described herein is administered once every two weeks (Q2W). In some embodiments, a liquid formulation or a unit dosage form as described herein is administered once every three weeks (Q3W). In some embodiments, a liquid formulation or a unit dosage form as described herein is administered once every four weeks (Q4W).

In some embodiments, Compound A is administered to a patient once every 1, 2, 3, 4, 5, 6, or 7 days. In some embodiments, a liquid formulation or a unit dosage form of the invention is administered to a patient biweekly (BIW). Biweekly doses can be administered hours apart (e.g., 1, 3, 6, 12 hours) or days apart (e.g., 1, 2, 3, or 4 days). In some embodiments, biweekly doses are administered on day 1 and day 2. In some embodiments, biweekly doses are administered on day 1 and day 2 on a Q3W dosing schedule. In some embodiments, biweekly doses are administered on day 1 and day 4. In certain embodiments, a liquid formulation or a unit dosage form as described herein is administered once per week (QW). In some embodiments, Compound A is intravenously administered is administered to a patient once every 1, 2, 3, or 4 weeks, or once every 7, 10, 14, 17, 21, 24, or 28 days.

As described herein in some embodiments, a liquid formulation or a unit dosage form is administered once weekly for one or two out of three weeks. In some embodiments, a liquid formulation or a unit dosage form as is administered twice weekly for one or two out of three weeks. In some embodiments, a liquid formulation or a unit dosage form is administered once weekly for one out of three weeks (e.g., on day 1 of a 21-day cycle). In some embodiments, a liquid formulation or a unit dosage form is administered twice weekly for one out of three weeks (e.g., on day 1 and 2 of a 21-day cycle).

As described herein in some embodiments, a liquid formulation or a unit dosage form is administered once weekly for one or two out of four weeks. In some embodiments, a liquid formulation or a unit dosage form as is administered twice weekly for one or two out of four weeks. In some embodiments, a liquid formulation or a unit dosage form is administered once weekly for one out of four weeks. In some embodiments, a liquid formulation or a unit dosage form is administered twice weekly for one out of four weeks. In some embodiments, a liquid formulation or a unit dosage form is administered once weekly every other week out of four weeks. In some embodiments, a liquid formulation or a unit dosage form is administered twice weekly every other week out of four weeks.

In some embodiments, a liquid formulation or a unit dosage form is administered to the patient once in week 1 in a 3 week administration cycle. In some embodiments, a liquid formulation or a unit dosage form is administered to the patient once in week 1 in a 4 week administration cycle. In some embodiments, a liquid formulation or a unit dosage form is administered to the patient once weekly in week 1 and week 2 in a 3 week administration cycle. In some embodiments, a liquid formulation or a unit dosage form is administered to the patient once weekly in week 1 and week 2 in a 4 week administration cycle. In some embodiments, a liquid formulation or a unit dosage form is administered to the patient once weekly in week 1 and week 3 in a 3 week administration cycle. In some embodiments, a liquid formulation or a unit dosage form is administered to the patient once weekly in week 1 and week 3 in a 4 week administration cycle. In some embodiments, a liquid formulation or a unit dosage form is administered to the patient once weekly in weeks 1-3 in a 4 week administration cycle. In some embodiments, a liquid formulation or a unit dosage form is administered to the patient once weekly in weeks 1-4 in a 4 week administration cycle (e.g., on days 1, 8, 15, and 22 of a 28-day cycle).

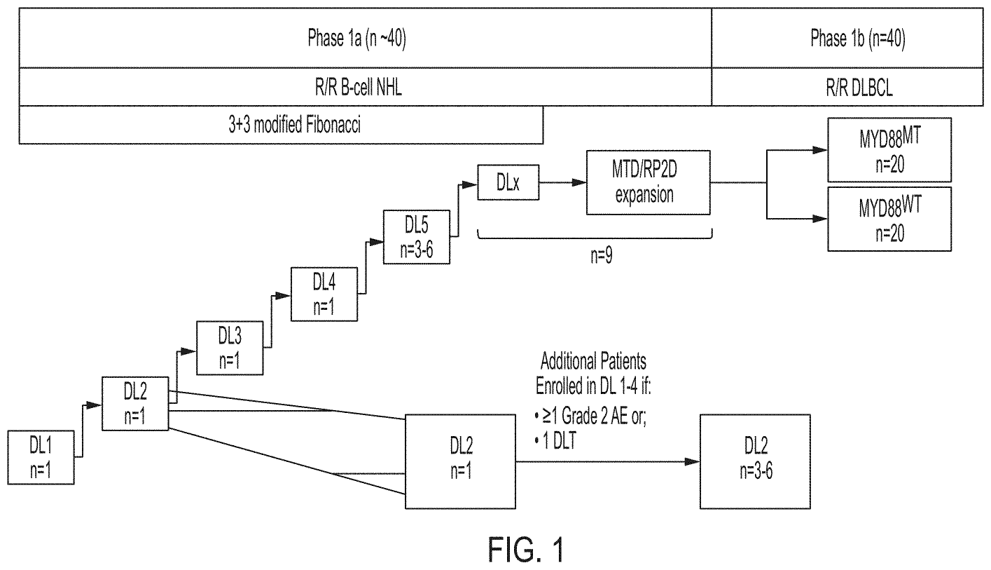

In some embodiments, a liquid formulation or a unit dosage form is administered to the patient twice in week 1 in a 3 week administration cycle. In some embodiments, a liquid formulation or a unit dosage form is administered to the patient twice in week 1 in a 4 week administration cycle. In some embodiments, a liquid formulation or a unit dosage form is administered to the patient twice weekly in week 1 and week 2 in a 3 week administration cycle. In some embodiments, a liquid formulation or a unit dosage form is administered to the patient twice weekly in week 1 and week 2 in a 4 week administration cycle. In some embodiments, a liquid formulation or a unit dosage form is administered to the patient twice weekly in week 1 and week 3 in a 4 week administration cycle. In some embodiments, a liquid formulation or a unit dosage form is administered to the patient twice weekly in weeks 1-3 in a 4 week administration cycle. In some embodiments, the dosing schedule shown in FIG. 1.

In some embodiments, an IV infusion of a unit dosage form of the invention lasts about 30-180 minutes (e.g., 60 min). In some embodiments, an IV infusion of a pharmaceutical composition of the invention lasts about 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 minutes, or any range of time created by using two of the aforementioned times as endpoints. In some embodiments, an IV infusion of a unit dosage form of the invention lasts about 30-90 minutes. In some embodiments, an IV infusion of a unit dosage form of the invention lasts about 60-120 minutes. In some embodiments, an IV infusion of a unit dosage form of the invention lasts about 1, 2, 2.5, or 3 hours. In some embodiments, an IV infusion of a unit dosage form of the invention lasts about 1 hour (e.g., 60 min).

In some embodiments, a method of the present invention comprises administering a liquid formulation or a unit dosage form as described herein, wherein a Cmax of up to about 5000 ng/mL of Compound A in plasma is achieved. In some embodiments, the administration of Compound A or a pharmaceutically acceptable salt thereof (e.g., in a liquid formulation or a unit dose form as described herein) achieves a Cmax of up to about 4000 ng/mL of Compound A in plasma. In some embodiments, the administration of Compound A or a pharmaceutically acceptable salt thereof (e.g., in a liquid formulation or a unit dose form as described herein) achieves a Cmax of up to about 3000 ng/mL of Compound A in plasma. In some embodiments, the administration of Compound A or a pharmaceutically acceptable salt thereof (e.g., in a liquid formulation or a unit dose form as described herein) achieves a Cmax of up to about 2000 ng/mL of Compound A in plasma.

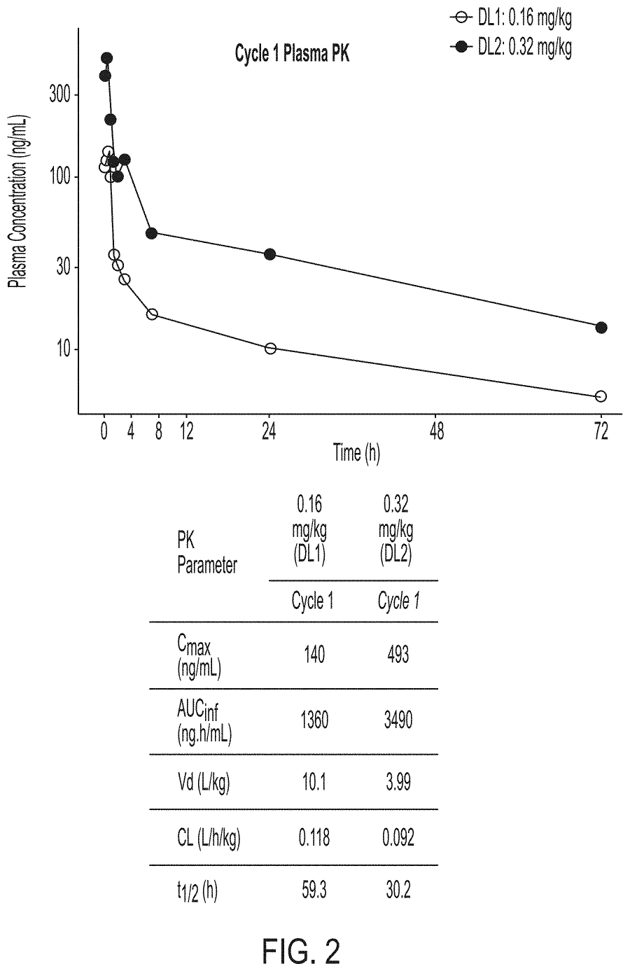

In some embodiments, a Cmax of Compound A in plasma includes about 100 ng/mL, 200 ng/mL, 300 ng/mL, 400 ng/mL, 500 ng/mL, 600 ng/mL, 700 ng/mL, 800 ng/mL, 900 ng/mL, 1000 ng/mL, 1100 ng/mL, 1200 ng/mL, 1300 ng/mL, 1400 ng/mL, 1500 ng/mL, 1600 ng/mL, 1700 ng/mL, 1800 ng/mL, 1900 ng/mL, 2000 ng/mL, 2100 ng/mL, 2200 ng/mL, 2300 ng/mL, 2400 ng/mL, 2500 ng/mL, 2600 ng/mL, 2700 ng/mL, 2800 ng/mL, 2900 ng/mL, 3000 ng/mL, 3100 ng/mL, 3200 ng/mL, 3300 ng/mL, 3400 ng/mL, 3500 ng/mL, 3600 ng/mL, 3700 ng/mL, 3800 ng/mL, 3900 ng/mL, 4000 ng/mL, 4100 ng/mL, 4200 ng/mL, 4300 ng/mL, 4400 ng/mL, 4500 ng/mL, 4600 ng/mL, 4700 ng/mL, 4800 ng/mL, 4900 ng/mL, and 5000 ng/mL, or any range of Cmax created by using two of the aforementioned concentrations as endpoints. In some embodiments, a Cmax of Compound A in plasma, as listed in FIG. 2, is achieved.

In some embodiments, the present disclosure provides a method of administering Compound A to a patient in need thereof, comprising administering to said patient a therapeutically effective amount of Compound A or a pharmaceutically acceptable salt thereof (e.g., in a liquid formulation or a unit dose form as described herein), wherein an AUC of up to about 10,000 ng*h/mL of Compound A in plasma is achieved. In some embodiments, the administration of Compound A or a pharmaceutically acceptable salt thereof (e.g., in a liquid formulation or a unit dose form as described herein) achieves an AUC of up to about 7500 ng*h/mL of Compound A in plasma. In some embodiments, the administration of Compound A or a pharmaceutically acceptable salt thereof (e.g., in a liquid formulation or a unit dose form as described herein) achieves an AUC of up to about 5000 ng*h/mL of Compound A in plasma.

In some embodiments, an AUC of Compound A in plasma includes about 1000 ng*h/mL, 1100 ng*h/mL, 1200 ng*h/mL, 1300 ng*h/mL, 1400 ng*h/mL, 1500 ng*h/mL, 1600 ng*h/mL, 1700 ng*h/mL, 1800 ng*h/mL, 1900 ng*h/mL, 2000 ng*h/mL, 2100 ng*h/mL, 2200 ng*h/mL, 2300 ng*h/mL, 2400 ng*h/mL, 2500 ng*h/mL, 2600 ng*h/mL, 2700 ng*h/mL, 2800 ng*h/mL, 2900 ng*h/mL, 3000 ng*h/mL, 3100 ng*h/mL, 3200 ng*h/mL, 3300 ng*h/mL, 2400 ng*h/mL, 2500 ng*h/mL, 2600 ng*h/mL, 2700 ng*h/mL, 2800 ng*h/mL, 2900 ng*h/mL, 3000 ng*h/mL, 3100 ng*h/mL, 3200 ng*h/mL, 3300 ng*h/mL, 3400 ng*h/mL, 3500 ng*h/mL, 3600 ng*h/mL, 3700 ng*h/mL, 3800 ng*h/mL, 3900 ng*h/mL, 4000 ng*h/mL, 4100 ng*h/mL, 4200 ng*h/mL, 4300 ng*h/mL, 4400 ng*h/mL, 4500 ng*h/mL, 4600 ng*h/mL, 4700 ng*h/mL, 4800 ng*h/mL, 4900 ng*h/mL, 5000 ng*h/mL, 5100 ng*h/mL, 5200 ng*h/mL, 5300 ng*h/mL, 5400 ng*h/mL, 5500 ng*h/mL, 5600 ng*h/mL, 5700 ng*h/mL, 5800 ng*h/mL, 5900 ng*h/mL, 6000 ng*h/mL, 6100 ng*h/mL, 6200 ng*h/mL, 6300 ng*h/mL, 6400 ng*h/mL, 6500 ng*h/mL, 6600 ng*h/mL, 6700 ng*h/mL, 6800 ng*h/mL, 6900 ng*h/mL, 7000 ng*h/mL, 7100 ng*h/mL, 7200 ng*h/mL, 7300 ng*h/mL, 7400 ng*h/mL, 7500 ng*h/mL, 7600 ng*h/mL, 7700 ng*h/mL, 7800 ng*h/mL, 7900 ng*h/mL, 8000 ng*h/mL, 8100 ng*h/mL, 8200 ng*h/mL, 8300 ng*h/mL, 8400 ng*h/mL, 8500 ng*h/mL, 8600 ng*h/mL, 8700 ng*h/mL, 8800 ng*h/mL, 8900 ng*h/mL, 9000 ng*h/mL, 9100 ng*h/mL, 9200 ng*h/mL, 9300 ng*h/mL, 9400 ng*h/mL, 9500 ng*h/mL, 9600 ng*h/mL, 9700 ng*h/mL, 9800 ng*h/mL, 9900 ng*h/mL, and 10,000 ng/mL, or any range of AUC created by using two of the aforementioned concentrations as endpoints. In some embodiments, an AUC of Compound A in plasma, as listed in FIG. 2, is achieved.

In some embodiments, a method of the present invention comprises administering a liquid formulation or a unit dosage form as described herein, wherein a Vd of up to about 20 L/kg of Compound A in plasma is achieved. In some embodiments, the administration of Compound A or a pharmaceutically acceptable salt thereof (e.g., in a liquid formulation or a unit dose form as described herein) achieves a Vd of up to about 10 L/kg of Compound A in plasma.

In some embodiments, a Vd of Compound A in plasma includes about 1 L/kg, 2 L/kg, 3 L/kg, 4 L/kg, 5 L/kg, 6 L/kg, 7 L/kg, 8 L/kg, 9 L/kg, 10 L/kg, 11 L/kg, 12 L/kg, 13 L/kg, 14 L/kg, 15 L/kg, 16 L/kg, 17 L/kg, 18 L/kg, 19 L/kg, and 20 L/kg, or any range of Vd created by using two of the aforementioned concentrations as endpoints. In some embodiments, a Vd of Compound A in plasma, as listed in FIG. 2, is achieved.

In some embodiments, a method of the present invention comprises administering a liquid formulation or a unit dosage form as described herein, wherein a CL of up to about 1 L/h/kg of Compound A in plasma is achieved. In some embodiments, the administration of Compound A or a pharmaceutically acceptable salt thereof (e.g., in a liquid formulation or a unit dose form as described herein) achieves a CL of up to about 0.5 L/h/kg of Compound A in plasma.

In some embodiments, a CL of Compound A in plasma includes about 0.01 L/h/kg, 0.02 L/h/kg, 0.03 L/b/kg, 0.04 L/h/kg, 0.05 L/b/kg, 0.06 L/b/kg, 0.07 L/h/kg, 0.08 L/h/kg, 0.09 L/h/kg, 0.1 L/h/kg, 0.11 L/h/kg, 0.12 L/h/kg, 0.13 L/h/kg, 0.14 L/b/kg, 0.15 L/h/kg, 0.16 L/h/kg, 0.17 L/h/kg, 0.18 L/b/kg, 0.19 L/h/kg, and 0.2 L/h/kg, or any range of CL created by using two of the aforementioned concentrations as endpoints. In some embodiments, a CL of Compound A in plasma, as listed in FIG. 2, is achieved.

In some embodiments, the present disclosure provides a method of administering Compound A to a patient in need thereof, comprising administering to said patient a therapeutically effective amount of Compound A or a pharmaceutically acceptable salt thereof (e.g., in a liquid formulation or a unit dose form as described herein), wherein a t½ of Compound A in plasma is from about 20 hrs to about 60 hrs. In some embodiments, the t½ of Compound A in plasma is from about 20 hrs to about 30 hrs, about 25 hrs to about 35 hrs, or about 30 hrs to about 40 hrs. In some embodiments, a t½ of Compound A in plasma, as listed in FIG. 2, is achieved.

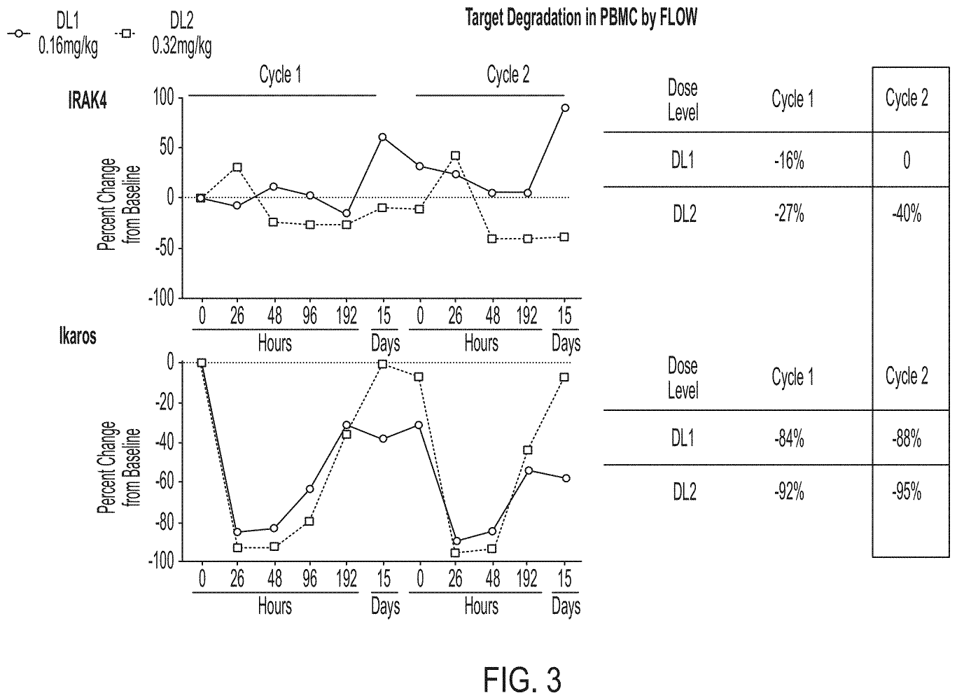

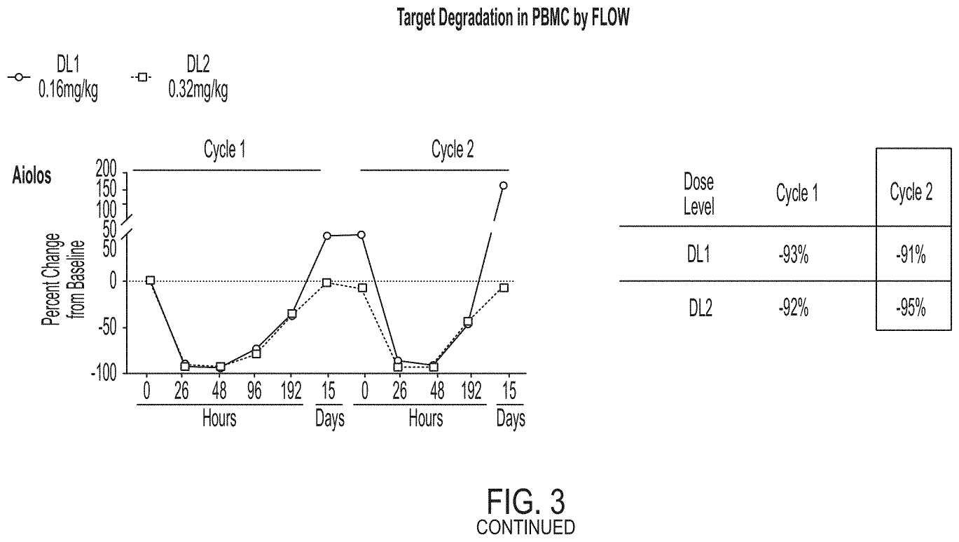

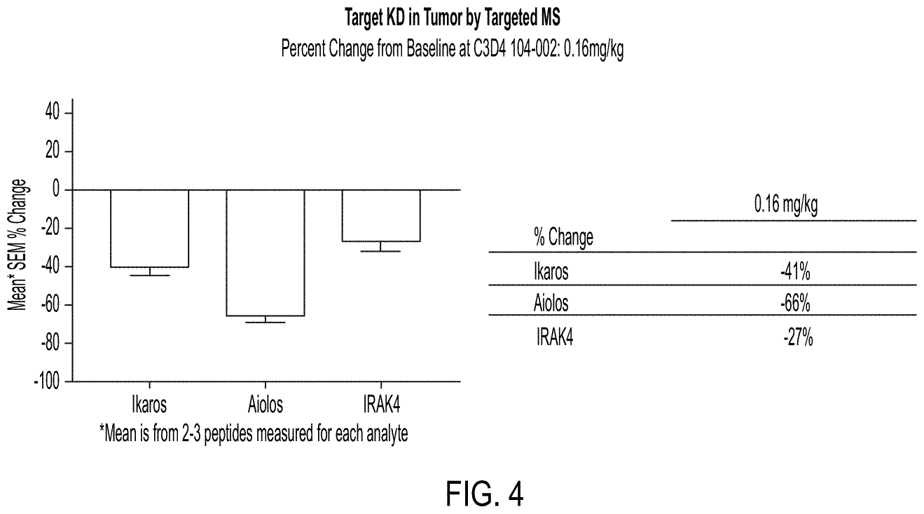

In some embodiments, the present disclosure provides a method of administering Compound A to a patient in need thereof, comprising administering to said patient a therapeutically effective amount of Compound A or a pharmaceutically acceptable salt thereof (e.g., in a liquid formulation or a unit dose form as described herein), wherein great than 30%, 40%, 50%, 60%, 70%, 80%, or 90% of IRAK4 degradation in blood or tumor is achieved (e.g., by measuring, at 48 hours post-administration, IRAK4 levels using mass spectrometry or lymphocytes and monocytes using flow cytometry). In some embodiments, an IRAK4 degradation, as listed in FIG. 3 or 4, is achieved.

In some embodiments, the present disclosure provides a method of administering Compound A to a patient in need thereof, comprising administering to said patient a therapeutically effective amount of Compound A or a pharmaceutically acceptable salt thereof (e.g., in a liquid formulation or a unit dose form as described herein), wherein greater than 40%, 50%, 60%, 70%, 80%, or 90% of Ikaros degradation in blood or tumor is achieved (e.g., by measuring, at 48 hours post-administration, Ikaros levels using mass spectrometry or lymphocytes and monocytes using flow cytometry). In some embodiments, an Ikaros degradation, as listed in FIG. 3 or 4, is achieved.

In some embodiments, the present disclosure provides a method of administering Compound A to a patient in need thereof, comprising administering to said patient a therapeutically effective amount of Compound A or a pharmaceutically acceptable salt thereof (e.g., in a liquid formulation or a unit dose form as described herein), wherein greater than 60%, 70%, 80%, or 90% of Aiolos degradation in blood or tumor is achieved (e.g., by measuring, at 48 hours post-administration, Aiolos levels using mass spectrometry or lymphocytes and monocytes using flow cytometry). In some embodiments, an Aiolos degradation, as listed in FIG. 3 or 4, is achieved.

It should also be understood that a specific dosage and treatment regimen for any particular patient will depend upon a variety of factors, including the activity of the specific compound employed, the age, body weight, general health, sex, diet, time of administration, rate of excretion, drug combination, drug metabolic capability, and the judgment of the treating physician and the severity of the particular disease being treated. The amount of a compound of the present invention in the composition will also depend upon the particular IRAKIMiD degrader in the composition.

### 4. Methods and Uses for Treating Disease

Compounds and compositions described herein are generally useful for the degradation of one or more enzymes. In some embodiments, the invention provides IRAKIMiD degraders that modulate targeted ubiquitination and degradation of IRAK4 kinase and IMiD substrates Ikaros and Aiolos.

Some of the most commonly employed E3 ligase ligands are thalidomide and its derivatives, lenalidomide and pomalidomide, commonly referred to as IMiDs (immunomodulatory imide drugs). These agents are small-molecule ligands of cereblon (CRBN) (Ito et al. “Identification of a primary target of thalidomide teratogenicity” Science 2010, 327(5971):1345-1350), a substrate adaptor for the ubiquitously expressed cullin ring ligase 4 (CUL4)-RBX1-DDB1-CRBN (CUL4CRBN) E3 ligase. It has been shown that thalidomide interacts with CRBN to form a novel surface, resulting in interactions with neosubstrates such as Ikaros (IKZF1) and Aiolos (IKZF3) and their ubiquitination and subsequent proteasomal degradation (Krönke et al. “Lenalidomide causes selective degradation of IKZF1 and IKZF3 in multiple myeloma cells” Science 2014, 343(6168):301-305; and Lu et al. “The myeloma drug lenalidomide promotes the cereblon-dependent destruction of Ikaros proteins” Science, 2014; 343(6168):305-309). This activity alone has potent antitumor effects in some liquid malignancies, and lenalidomide (Revlimid®) is US Food and Drug Administration approved for the treatment of MCL, multiple myeloma, and myelodysplastic syndromes with deletion of chromosome 5q. Lenalidomide is also undergoing late-stage clinical trials for a number of lymphomas, including MCL and the activated B-cell subtype of diffuse large B-cell lymphoma (ABC DLBCL).

It has been shown that activating MYD88 mutations increase production of beta-IFN, a pro-apoptotic cytokine, in ABC-DLBCL cells (Yang et al. “Exploiting synthetic lethality for the therapy of ABC diffuse large B cell lymphoma” Cancer Cell 2012, 21(6):723-737). The cells are rendered resistant to this effect by a concomitant MYD88-driven activation of NFkB signaling via IRF4 and SPIB transactivating CARD11 (Yang, Cancer Cell 2012). IMiDs are also known to increase the IFN response in MYD88 mutant ABC-DLBCL to levels sufficient to increase apoptosis (Yang, Cancer Cell 2012; and Hagner et al. “CC-122, a pleiotropic pathway modifier, mimics an interferon response and has antitumor activity in DLBCL” Blood 2015, 126:779-789). This effect has been shown to synergize with inhibition of NFkB signaling to further drive DLBCL cell death (Yang, Cancer Cell 2012).

In some instances, the combination of an IMiD with a small molecule IRAK4 kinase inhibitor shows little to no additive effect on viability of the MYD88 mutant ABC DLBCL cell lines, such as OCI-LY10. In some embodiments, the combination of an IRAK4 inhibitor with IMiD is less active than the IRAKIMiD degraders provided herein.

In certain embodiments, the combination of IRAK4 degradation with IKZF1 and IKZF3 degradation shows potent, single agent activity versus MYD88 mutant ABC DLBCL cell lines in vitro and OCI-LY10 xenograft in vivo. In some embodiments, IRAKIMiD retain degradation of Ikaros (IKZF1) and other known IMiDs neosubstrates, while more strongly inducing an interferon response compared to pomalidomide alone. In some embodiments, IRAKIMiD degraders are potent at killing MYD88 mutant ABC DLBCL cell lines in vitro, demonstrating increased activity versus that obtained from combining an IRAK4 inhibitor with IMiDs as single agents.

In certain embodiments, a provided IRAKIMiD degraders degrades IRAK4, Ikaros, and Aiolos in MYD88 mutant ABC DLBCL cell line xenografts in vivo, and strongly induces a signature of interferon-driven proteins exemplified by IFIT1 (interferon-inducible transcript 1) and IFIT3 (interferon-inducible transcript 3). In some embodiments, a provided IRAKIMiD degrader drives regression of tumor xenografts as a single agent.

In some embodiments, the provided compounds of present invention highlight a synergy obtained by combining IRAK4 degradation with IMiD induction of interferon response to drive single agent anti-tumor activity in MYD88 mutant DLBCL and possibly in other heme malignancies. In certain embodiments, a provided IRAKIMiD degrader degrades IRAK4, Ikaros, and Aiolos, acts synergistically. In some embodiments, a provided IRAKIMiD degrader degrades IRAK4, Ikaros, and Aiolos with increased activity in comparison to a provided IRAKIMiD degrader comprising the same IRAK4 binder and a non-IMiD-based E3 ligase and the same IMiD-based E3 ligase as a single agent.

In some embodiments, the proliferative disease which can be treated according to the methods of this invention is an MyD88 driven disorder. In some embodiments, the MyD88 driven disorder which can be treated according to the methods of this invention is selected from ABC DLBCL, primary central nervous system (CNS) lymphomas, primary extranodal lymphomas, Waldenström macroglobulinemia, Hodgkin's lymphoma, primary cutaneous T-cell lymphoma, and chronic lymphocytic leukemia (CLL). In some embodiments, the proliferative disease which can be treated according to the methods of this invention is a mutant MyD88 disorder. In some embodiments, the proliferative disease which can be treated according to the methods of this invention is a wild-type MyD88 disorder.

In some embodiments, the present disclosure provides a method for treating a relapsed and/or refractory B-cell non-Hodgkin lymphoma in a patient, comprising administering to the patient a therapeutically effective amount of Compound A, or a pharmaceutically acceptable salt thereof, or a liquid formulation thereof as described herein.

In some embodiments the relapsed and/or refractory B-cell non-Hodgkin lymphoma which can be treated according to the methods of this invention is selected from diffuse large B-cell lymphoma (DLBCL), ABC DLBCL, primary mediastinal B-cell lymphoma, primary extranodal lymphomas, primary CNS lymphoma, primary cutaneous large B-cell lymphoma, follicular lymphoma, chronic lymphocytic leukemia (CLL), small lymphocytic lymphoma (SLL), mantle cell lymphoma (MCL), marginal zone lymphomas, nodal marginal zone B-cell lymphoma, splenic marginal zone B-cell lymphoma, extranodal marginal zone B-cell lymphoma, mucosa-associated lymphoid tissue (MALT) lymphoma, Burkitt lymphoma, Waldenström macroglobulinemia, hairy cell leukemia (HCL), and primary intraocular lymphoma.

In some embodiments, the present disclosure provides a method for treating a MYD88-mutant B-cell lymphoma in a patient, comprising administering to the patient a therapeutically effective amount of Compound A, or a pharmaceutically acceptable salt thereof, or a liquid formulation thereof as described herein.

In some embodiments, the present invention provides a method of treating DLBCL in a patient in need thereof, comprising administering an IRAKIMiD degrader (e.g., Compound A) of the present invention, or a pharmaceutically acceptable salt thereof.

In some embodiments, the present invention provides a method of treating ABC DLBCL in a patient in need thereof, comprising administering an IRAKIMiD degrader (e.g., Compound A) of the present invention, or a pharmaceutically acceptable salt thereof.

In some embodiments, the present invention provides a method of treating primary mediastinal B-cell lymphoma in a patient in need thereof, comprising administering an IRAKIMiD degrader (e.g., Compound A) of the present invention, or a pharmaceutically acceptable salt thereof.

In some embodiments, the present invention provides a method of treating primary extranodal lymphomas DLBCL in a patient in need thereof, comprising administering an IRAKIMiD degrader (e.g., Compound A) of the present invention, or a pharmaceutically acceptable salt thereof.

In some embodiments, the present invention provides a method of treating primary CNS lymphoma DLBCL in a patient in need thereof, comprising administering an IRAKIMiD degrader (e.g., Compound A) of the present invention, or a pharmaceutically acceptable salt thereof.

In some embodiments, the present invention provides a method of treating primary cutaneous large B-cell lymphoma in a patient in need thereof, comprising administering an IRAKIMiD degrader (e.g., Compound A) of the present invention, or a pharmaceutically acceptable salt thereof.

In some embodiments, the present invention provides a method of treating follicular lymphoma in a patient in need thereof, comprising administering an IRAKIMiD degrader (e.g., Compound A) of the present invention, or a pharmaceutically acceptable salt thereof.

In some embodiments, the present invention provides a method of treating chronic lymphocytic leukemia (CLL) in a patient in need thereof, comprising administering an IRAKIMiD degrader (e.g., Compound A) of the present invention, or a pharmaceutically acceptable salt thereof.

In some embodiments, the present invention provides a method of treating small lymphocytic lymphoma (SLL) in a patient in need thereof, comprising administering an IRAKIMiD degrader (e.g., Compound A) of the present invention, or a pharmaceutically acceptable salt thereof.

In some embodiments, the present invention provides a method of treating mantle cell lymphoma (MCL) in a patient in need thereof, comprising administering an IRAKIMiD degrader (e.g., Compound A) of the present invention, or a pharmaceutically acceptable salt thereof.

In some embodiments, the present invention provides a method of treating marginal zone lymphomas in a patient in need thereof, comprising administering an IRAKIMiD degrader (e.g., Compound A) of the present invention, or a pharmaceutically acceptable salt thereof.

In some embodiments, the present invention provides a method of treating nodal marginal zone B-cell lymphoma in a patient in need thereof, comprising administering an IRAKIMiD degrader (e.g., Compound A) of the present invention, or a pharmaceutically acceptable salt thereof.

In some embodiments, the present invention provides a method of treating splenic marginal zone B-cell lymphoma in a patient in need thereof, comprising administering an IRAKIMiD degrader (e.g., Compound A) of the present invention, or a pharmaceutically acceptable salt thereof.

In some embodiments, the present invention provides a method of treating extranodal marginal zone B-cell lymphoma in a patient in need thereof, comprising administering an IRAKIMiD degrader (e.g., Compound A) of the present invention, or a pharmaceutically acceptable salt thereof.

In some embodiments, the present invention provides a method of treating mucosa-associated lymphoid tissue (MALT) lymphoma in a patient in need thereof, comprising administering an IRAKIMiD degrader (e.g., Compound A) of the present invention, or a pharmaceutically acceptable salt thereof

In some embodiments, the present invention provides a method of treating Burkitt lymphoma, Waldenström macroglobulinemia in a patient in need thereof, comprising administering an IRAKIMiD degrader (e.g., Compound A) of the present invention, or a pharmaceutically acceptable salt thereof

In some embodiments, the present invention provides a method of treating hairy cell leukemia (HCL) in a patient in need thereof, comprising administering an IRAKIMiD degrader (e.g., Compound A) of the present invention, or a pharmaceutically acceptable salt thereof.

In some embodiments, the present invention provides a method of treating primary intraocular lymphoma in a patient in need thereof, comprising administering an IRAKIMiD degrader (e.g., Compound A) of the present invention, or a pharmaceutically acceptable salt thereof.

In some embodiments, the present invention provides a method of treating nodular lymphocyte-predominant Hodgkin lymphoma in a patient in need thereof, comprising administering an IRAKIMiD degrader (e.g., Compound A) of the present invention, or a pharmaceutically acceptable salt thereof

In some embodiments the proliferative disease which can be treated according to the methods of this invention is an IL-1 driven disorder. In some embodiments the IL-1 driven disorder is Smoldering of indolent multiple myeloma.

In some embodiments, the present invention provides a method for the treatment of adult patients with a relapsed and/or refractory B-cell non-Hodgkin lymphoma who have received one prior therapy.

In some embodiments, the present invention provides a method for the treatment of adult patients with a relapsed and/or refractory B-cell non-Hodgkin lymphoma who have received two prior therapies.

In some embodiments, the present invention provides a method for the treatment of adult patients with a relapsed and/or refractory B-cell non-Hodgkin lymphoma who have received three prior therapies.

In some embodiments, the present invention provides a method for the treatment of adult patients with a relapsed and/or refractory B-cell non-Hodgkin lymphoma who have received at least one prior therapy.

In some embodiments, the present invention provides a method for the treatment of adult patients with a relapsed and/or refractory B-cell non-Hodgkin lymphoma who have received at least two prior therapies.

In some embodiments, the present invention provides a method for the treatment of adult patients with a relapsed and/or refractory B-cell non-Hodgkin lymphoma who have received at least three prior therapies.

The following examples are provided for illustrative purposes only and are not to be construed as limiting this invention in any manner.

### EXEMPLIFICATION

List of Abbreviations

AE Adverse event

ALCL Anaplastic large cell lymphoma

ALT Alanine aminotransferase

ANC Absolute neutrophil count

AST Aspartate transaminase

AUC Area under the concentration-time curve

BSA Body surface area

CHOP Cyclophosphamide, doxorubicin, vincristine, and prednisone

CL Apparent total body clearance

Cmax Maximum plasma drug concentration

CNS Central nervous system

COVID-19 Coronavirus disease 2019

CR Complete response

CRBN Cereblon

eCRF Electronic case report form

CRO Contract research organization

CT Computed tomography

CTCL Cutaneous T-cell lymphoma

ctDNA Circulating tumor DNA

CYP Cytochrome P450

C1D1 Cycle 1 Day 1

C2D1 Cycle 2 Day 1

DCR Disease control rate

DDI Drug-drug interaction

DLT Dose-limiting toxicity

DNA Deoxyribonucleic acid

DOR Duration of response

DRF Dose range finding

ECG Electrocardiogram

ECOG Eastern Cooperative Oncology Group

EDC Electronic data capture

EMA European Medicine Agency

OI End of infusion

EOT End of treatment

FDA Food and Drug Administration

fe Fraction excreted/recovered in urine

FFPE Formalin-fixed paraffin-embedded

FFS Failure-free survival

FIH First-in-human

FSH Follicle-stimulating hormone

GCP Good Clinical Practice

GLP Good Laboratory Practice

HbcAb Hepatitis C core antibody

HbsAg Hepatitis B surface antigen

HCV Hepatitis C virus

HIV Human immunodeficiency virus

HRT Hormonal replacement therapy

IB Investigator's Brochure

ICF Informed consent form

ICH International Conference for Harmonisation

IEC Independent Ethics Committee

IMiD Immunomodulatory imide drug

INR International normalized ratio

IRB Institutional Review Board

IV Intravenous

JAK Janu kinase

LAR Legally authorized representative

LGL-L Large Granular Lymphocyte Leukemia

LHRH Luteinizing hormone-releasing hormone

MAD Maximum administered dose

MF Mycosis fungoides

MRI Magnetic resonance imaging

mSWAT Modified severity-weighted assessment tool

MTD Maximum tolerated dose

NCI TCAE National Cancer Institute Common Terminology Criteria for Adverse Events

NHL Non-Hodgkin Lymphoma

NK Natural killer

NTL Non-target Lesions

OR Objective response

ORR Objective response rate

OS Overall survival

PBMC Peripheral blood mononuclear cell

PD Pharmacodynamic(s)

PET Positron emission tomography

PFS Progression-free survival

PK Pharmacokinetic(s)

PR Partial response

PTCL Peripheral T-cell lymphoma

PTCL-NOS PTCL-not otherwise specified

q.s. Quantum sufficit (“as much as sufficient”)

QTcF QT interval corrected by Fridericia's formula

QW Once weekly

RBC Red blood cell

RECIST Response evaluation criteria in solid tumors

RP2D Recommended Phase 2 dose

R/R Relapsed/refractory

SAE Serious adverse event

SAP Statistical Analysis Plan

SARS-CoV-2 Severe acute respiratory syndrome coronavirus 2

SRC Safety Review Committee

SS Sézary syndrome

STAT Signal transducers and activators of transcription

SUSAR Suspected unexpected serious adverse reaction

t½ Elimination half-life

TEAE Treatment-emergent adverse event

TL Target lesion

tmax Time to reach Cmax following drug administration

LN Upper limit of normal

UPS Ubiquitin-proteosome system

US United States

Vd Apparent volume of distribution

Vdss Volume of distribution at steady state

WHO World Health Organization

WHODD World Health Organization Drug Dictionary

WOCBP Woman of childbearing potential

### EXAMPLE 1

### Synthesis of Compound A

Compound A can be prepared by methods known to one of ordinary skill in the art, for example, as described in WO 2021/127190, the contents of which are incorporated herein by reference in their entireties.

### EXAMPLE 2

### A Phase 1, Multicenter, Open-Label, Dose Escalation and Expansion Study to Evaluate the Safety, Tolerability, Pharmacokinetics, Pharmacodynamics, and Clinical Activity of Intravenously Administered Compound A in Adult Patients with Relapsed or Refractory B-cell Non-Hodgkin Lymphoma

Rationale: Diffuse large B-cell lymphoma (DLBCL) are thought to represent about 30% of all cases of non-Hodgkin lymphoma (NHL). Approximately 35% to 40% of patients with DLBCL have disease that relapses after or is refractory to first-line therapy and generally have poor outcomes. None of the available therapies for treating relapsed/refractory (R/R) DLBCL are considered curative and all have distinct toxicities highlighting the need for novel therapies.

Compound A is a potent, highly selective, heterobifunctional small molecule degrader of interleukin-1 receptor-associated kinase 4 (IRAK4) and the immunomodulatory imide drugs (IMiDs) substrates Ikaros and Aiolos. This first-in-human (FIH) study is aimed at evaluating the overall safety profile of escalating doses of Compound A and to determine the maximum tolerated dose (MTD) and the recommended Phase 2 dose (RP2D) of Compound A in patients with R/R B-cell NHL.

Objectives and Endpoints:

Phase 1a

| Objectives | Endpoints |
|---|---|
| Primary | Incidence and severity of adverse events (Aes) |
| To evaluate the overall safety profile of | graded according to the National Cancer Institute |
| escalating doses of Compound A and to | Common Terminology Criteria for Adverse |
| determine the maximum tolerated dose (MTD) | Events (NCI CTCAE) version 5.0, clinical |
| and the recommended Phase 2 dose (RP2D) of | laboratory abnormalities, and electrocardiogram |
| Compound A | (ECG) abnormalities |
| Secondary | PK parameters for Compound A and its |
| To characterize the pharmacokinetics (PK) of | enantiomers Compound I-A and Compound (S)- |
| Compound A and its enantiomers | A (and potential metabolites) derived from |
| | plasma and urine concentrations |
| To obtain preliminary estimates of clinical | Objective Response Rate (ORR) based on |
| activity of Compound A | Investigator's Assessment as per Lugano 2014 |
| | criteria and Duration of Response (DOR) |
| Exploratory | MYD88 gain of function mutation analysis in |
| To evaluate the relationship between baseline | tumor and circulating tumor DNA (ctDNA) |
| MYD88 mutation status and response to | |
| Compound A | |
| To evaluate the relationship between baseline | Somatic mutation analysis of tumor tissue |
| tumor genotype and response to Compound A | |
| To assess the pharmacodynamic (PD) effects of | Protein analysis of tumor tissue and peripheral |
| Compound A | blood mononuclear cells (PBMCs) in pre- and |
| | post-treatment samples |

Phase 1b

| Objectives | Endpoints |
|---|---|
| Primary | Incidence and severity of adverse events (Aes) |
| To evaluate the safety and tolerability of | graded according to the National Cancer Institute |
| Compound A at the recommended Phase 2 dose | Common Terminology Criteria for Adverse |
| (RP2D) in patients with MYD88 mutant | Events (NCI CTCAE), version 5.0, and changes |
| (MYD88-MT) and MYD88 wild type (MYD88- | in clinical laboratory parameters, vital signs, and |
| WT) relapsed/refractory diffuse large B-cell | electrocardiograms (ECGs) |
| lymphoma (DLBCL) | |
| Secondary | Objective Response Rate (ORR), Duration of |
| To obtain preliminary estimates of clinical | Response (DOR), Progression-free Survival |
| activity of Compound A in adult patients with | (PFS), Disease control rate (DCR), and Overall |
| MYD88-MT and MYD88-WT | Survival (OS) |
| relapsed/refractory DLBCL | |
| To characterize the pharmacokinetics (PK) of | PK parameters for Compound A and its |
| Compound A and its enantiomers in plasma | enantiomers Compound I-A and Compound (S)- |
| | A derived from plasma concentrations |
| Exploratory | MYD88 gain of function mutation analysis in |
| To evaluate the relationship between baseline | tumor and circulating tumor DNA (ctDNA) |
| MYD88 mutation status and response to | |
| Compound A | |
| To evaluate the relationship between tumor | Somatic mutation analysis of tumor tissue |
| genotype and response to Compound A | |
| To assess the pharmacodynamic (PD) effects of | Protein analysis of tumor tissue and peripheral |
| Compound A in MYD88-MT and MYD88-WT | blood mononuclear cells (PBMCs) in pre- and |
| relapsed/refractory DLBCL patients | post-treatment samples |

Overall Design: This FIH study is an open-label Phase 1a/1b dose escalation and dose expansion study in adult patients with R/R B-cell NHL.

Patients who provide informed consent and meet the eligibility criteria for the study will be enrolled and treated with Compound A administered intravenously (IV) on Day 1 of a 21-day schedule (Schedule 1). An alternative schedule of Compound A administered IV on Day 1 and Day 2 of a 21-day schedule (Schedule 2) may also be evaluated based on emerging pharmacokinetic, pharmacodynamic, and safety data observed in Schedule 1. If Schedule 2 is investigated, the first dose level will represent a split dose of the highest dose previously determined to be tolerable in Schedule 1 with the total dose administered in Schedule 2 being equivalent to the total dose administered in Schedule 1. Subsequent dose levels for Schedule 2 will follow the same dose escalation scheme as described for Schedule 1. If the sponsor and investigators determine that, based on the observed safety, PK, preliminary efficacy, and/or the PD profile, Schedule 2 may not be beneficial to patients, then the study will proceed immediately to Phase 1b after the Schedule 1 MTD/RP2D is determined and Schedule 2 may not be evaluated.
Schedule 1: Dose D1 on a 21-day schedule
Schedule 2: Dose D1, D2 on a 21-day schedule

Patients will remain on study treatment until disease progression, unacceptable toxicity, withdrawal of consent, any study-specific discontinuation criteria are met, or the Investigator determines that it is in the best interest of the patient to discontinue study treatment. One on-treatment biopsy will be required in Phase 1b unless medically contraindicated or is unattainable due to lack of feasibility. This biopsy will be optional in Phase 1a. An additional biopsy at time of disease progression will be optional. The end of treatment/safety follow-up visit will be scheduled within 30 days from the last dose of Compound A. Further, patients will be contacted every 3 months to collect data on survival status and subsequent therapies for up to one year after their last dose.

The study will be conducted in 2 parts: dose escalation with MTD/RP2D confirmation (Phase 1a) and dose expansion (Phase 1b). Up to 40 evaluable patients will be enrolled in Phase 1a; the total number of patients will depend on the number of dose levels explored. Up to 20 evaluable patients will be enrolled in each of the 2 cohorts of MYD88-MT and MYD88-WT tumors in Phase 1b. The study schema is provided in FIG. 1.

Phase 1a: This part aims to characterize the safety and tolerability of ascending doses of Compound A in patients with R/R B-cell NHL. The objective is to define the MTD and RP2D. Approximately 8 dose levels of Compound A are planned to be evaluated: 0.16 mg/kg, 0.32 mg/kg, 0.64 mg/kg, 1.25 mg/kg, 2.0 mg/kg, 3.0 mg/kg, 4.2 mg/kg, and 5.6 mg/kg. The escalation cohort dose levels and safety of dose escalation for ongoing patients will be determined by the Safety Review Committee (SRC) based on the review of all available data including, but not limited to safety and PK, as guided by the dose escalation rules. Once MTD/RP2D is determined in a cohort of 3-6 patients, it will be confirmed by enrolling additional patients with B-cell NHL in this cohort until a total of 9 patients are enrolled prior to initiation of Phase lb.

Phase 1b, Dose Expansion: After the completion of Phase 1a, up to 40 additional patients with R/R DLBCL will be treated at the RP2D in the following cohorts, to further characterize tolerability of the RP2D and to evaluate the relative clinical activity of Compound A in adult patients with MYD88-MT and MYD88-WT R/R DLBCL:
Cohort 1: MYD88-MT R/R DLBCL (n =up to 20)
Cohort 2: MYD88-WT R/R DLBCL (n =up to 20)

Patient's safety will be monitored throughout the study by the SRC established by the Sponsor. This committee will monitor all treatment-emergent data (e.g., pharmacokinetics [PK], safety [including, but not limited to dose limiting toxicities (DLTs)]) on an ongoing basis to ensure the continued safety of patients enrolled in this study. Cumulative data will be monitored for any late onset toxicities.

### Study Population

Inclusion criteria. Patients are eligible to be included in the study only if all of the following criteria apply:
1. Male or female aged ≥18 years on the day of signing the informed consent.
2. Phase 1a Only: Histologically confirmed diagnosis of B-cell NHL according to the World Health Organization (WHO) classification. A patient with evidence of histological transformation of indolent lymphoma to DLBCL is also eligible.
3. Phase 1a Only: Fresh/archival formalin fixed paraffin embedded (FFPE) tumor tissue preferably collected within 6 months prior to first dose (C1D1) and a blood sample for central testing of MYD88 mutational analysis must be available. If tumor tissue or blood sample is unavailable, discussion with the Medical Monitor is required prior to enrollment.
4. Phase 1b Only: Histologically confirmed diagnosis of DLBCL according to the WHO classification.

A patient with evidence of histological transformation to DLBCL from an earlier diagnosis of low-grade lymphoma with subsequent DLBCL relapse is also eligible.
5. Phase 1b Only: Documented tumor MYD88 status (as mutant or wild type). Either freshly collected or archival (collected within 6 months prior to first dose [C1D1]) FFPE tumor and pre-treatment blood sample must be submitted for determination of MYD88 status (as mutant or wild type).
6. Disease relapsed and/or refractory to at least 1 accepted standard systemic regimen.
   Note: Patients with R/R disease with only 1 line of prior systemic therapy can be considered for enrollment only if:
      in the opinion of the Investigator, the patient is not a good candidate or is not currently considered eligible for autologous stem cell transplantation (ASCT) or CAR-T therapy, or
      patient has refused ASCT or CAR-T therapy.
   Note: Prior ASCT or CAR-T therapy are not exclusionary.
7. At least one bi-dimensionally measurable disease site. The lesion must have a greatest transverse diameter of at least 1.5 cm and greatest perpendicular diameter of at least 1.0 cm at Screening. The lesion must be positive on positron emission tomography (PET) scan. Patients with non-measurable but evaluable disease are eligible for Phase 1a, cohorts 1-4.
8. Eastern Cooperative Oncology Group (ECOG) performance status of 0-2 at Screening.
9. Adequate organ and hematologic function at Screening and on C1D1 (pre-dose) defined as:
   Hematology
      absolute neutrophil count (ANC)≥1000 μL)
      hemoglobin≥8.0 g/dL (for those patients undergoing red blood cell [RBC] transfusion, hemoglobin must be evaluated at least 14 days after the last RBC transfusion)
      platelet count≥75,000/μL (for those patients undergoing transfusion, platelet count must be evaluated at least 7 days after the last platelet transfusion)
   Hepatic Function
      aspartate aminotransferase (AST), alanine transaminase (ALT)≤3× upper limit of normal (ULN) or <5× ULN in cases of documented lymphoma involvement of liver
      total serum bilirubin≤1.5× ULN or <5× ULN if secondary to Gilbert's syndrome or documented lymphoma involvement of liver
   Renal Function
      serum electrolyte (potassium, calcium, and magnesium) levels within the normal reference range (may be supplemented according to institutional standard)
      serum creatinine clearance≥60 mL/min/1.73 m² either measured or calculated using standard Cockcroft-Gault formula.
10. Negative SARS-CoV-2 test at Screening.
11. Women of childbearing potential (WOCBP) must agree to use two highly effective contraceptive methods for the duration of study treatment and 6 months after the last dose of study medication.
12. WOCBP must have a negative serum pregnancy test at Screening and within 72 hours prior to first dose of the study drug.
13. Men must agree to use two highly effective contraceptive methods during the study treatment and for 6 months after the last dose of study medication if the partner is a WOCBP.
14. Patient understands signed and dated, written informed consent and provides voluntary consent prior to any mandatory study-specific procedures, sampling, and analyses. Patient is capable of giving signed informed consent which includes compliance with the requirements and restrictions listed in the informed consent form (ICF) and in this protocol.

Exclusion Criteria. Patients are excluded from the study if any of the following criteria apply:
1. Known central nervous system (CNS) lymphoma or meningeal involvement.
2. History of malignancy other than B-cell NEIL or DLBCL unless the patient has been disease-free for ≥2 years. Exceptions to the ≥2-year time limit include treated basal cell or localized squamous cell skin carcinoma, localized prostate cancer, or other localized carcinomas such as carcinoma in situ of cervix, breast, or bladder.
3. Active concurrent malignancy with the exception of basal cell or localized squamous cell skin carcinoma, localized prostate cancer, or other localized carcinomas such as carcinoma in situ of cervix, breast, or bladder.
4. Patient has not recovered from any clinically significant adverse events (Aes) of previous treatments to pre-treatment baseline or Grade 1 prior to first dose of study drug.
5. Ongoing unstable cardiovascular function:
   Symptomatic ischemia, or
   Uncontrolled clinically significant conduction abnormalities (i.e., ventricular tachycardia on anti-arrhythmia are excluded; 1st degree atrioventricular block or asymptomatic left anterior fascicular block/right bundle branch block will not be excluded), or
   Congestive heart failure of New York Heart Association Class≥III, or
   Myocardial infarction within 3 months prior to Screening.
6. Congenital long QT syndrome, or a QT interval corrected by Fridericia's formula (QTcF)≥450 ms (average of triplicate ECGs) at Screening and/or on C1D1 (pre-dose) with the exception of a documented bundle branch block or unless secondary to pacemaker. In the case of a documented bundle branch block or a pacemaker, discussion with the Medical Monitor is required prior to enrollment.
7. Thromboembolic or cerebrovascular event (i.e., transient ischemic attacks, cerebrovascular accidents, pulmonary emboli, or clinically significant deep vein thrombosis)≤6 months prior to first dose of study drug.
8. Infection requiring antibiotics, antivirals, or antifungals within 1 week prior to first dose of study drug, unless such infection is adequately controlled (defined as exhibiting no ongoing signs/symptoms related to the infection and with clinical improvement). In the case of prophylactic use of these agents, discussion with the Medical Monitor is required prior to enrollment.
9. Positive hepatitis B and/or hepatitis C serology or known seropositivity for or history of active viral infection with human immunodeficiency virus (HIV).
10. Concurrent medical conditions including psychiatric disorders that in the judgment of the Investigator will interfere with the patient's ability to participate or with achieving the objectives of the study or pose a safety risk.
11. Patient is pregnant or breast feeding.
12. Prior treatment with an IRAK4 inhibitor.
13. Disease progression on IMiD-containing regimen <6 months prior to first dose of study drug.
14. Relapsed/refractory disease after ≥2 prior IMiD-containing regimens.
15. Discontinuation of prior IMiD therapy due to IMiD-related toxicity.
16. Prior allogeneic hematopoietic stem cell transplant.
17. Autologous hematopoietic stem cell transplant within 6 months prior to first dose of study drug or patient has progressed within 6 months from the day of stem cell infusion.
18. Radiation treatment within 4 weeks prior to first dose of study drug, unless the tumor site continues to increase in size after the patient has completed radiotherapy treatment.
19. Major surgery requiring general anesthesia within 4 weeks prior to first dose of study drug. If patient required general anesthesia within the prior 4 weeks, consultation with the Medical Monitor is required prior to enrollment.
20. Received live vaccine within 1 month prior to the first dose of study drug.
21. Exposure to investigational or non-investigational anti-cancer therapy within 2 weeks or within at least 4 half-lives (up to a maximum of 4 weeks) prior to the first dose of study drug, whichever is longer. In all situations, the maximum washout period will not exceed 4 weeks prior to first dose of study drug. Note: Low dose steroids (oral prednisone or equivalent ≤20 mg/day), localized non-CNS radiotherapy, previous hormonal therapy with luteinizing hormone-releasing hormone (LHRH) agonists for prostate cancer, and treatment with bisphosphonates and RANKL inhibitors are not criteria for exclusion.
22. Patient has completed a course of SARS-CoV-2 vaccine within 14 days prior to first dose of study drug.
23. Patient is unable or unwilling to discontinue prohibited concomitant medications or adhere to restrictions for use of concomitant medications.
24. Concurrent medical conditions including psychiatric disorders that in the judgment of the Investigator will interfere with the patient's ability to participate or with achieving the objectives of the study or pose a significant safety risk.
25. Patient is unable or unwilling to comply with all requirements of the study.
26. Person who has been committed to an institution by official or judicial order.
27. Patient with dependency on the Sponsor, Investigator or study site.

### Statistical Considerations

No formal statistical hypotheses will be tested in this dose escalation and dose expansion, single treatment group study. Safety, efficacy, PK, and pharmacodynamics (PD) assessments will be summarized separately for the dose escalation and dose expansion portions of the study. Additional summaries of pooled data across cohorts and/or dose groups may also be generated. Descriptive and summary statistics will be presented for the assessments and will include number of observations, mean, standard deviation, median, and range for continuous variables while categorical data will be summarized using frequency counts and percentages. Listings and graphical summaries of the data may be presented. All details of the data summaries and displays will be presented in a formal Statistical Analysis Plan (SAP) which will be finalized prior to final database lock.

### Preliminary Phase 1a Results

FIG. 2 depicts plasma concentration and PK in DL1 and DL2 showing a dose-proportional increase in exposure.

FIGS. 3 and 4 shows the degradation profile of IRAK4, Ikaros, and Aiolos is consistent with preclinical models in blood and tumor. Up to 40% KD of IRAK4 and 95% KD of Ikaros and Aiolos was demonstrated in PBMC in DL1 and DL2. 27% IRAK4 KD and 40-66% Ikaros and Aiolos KD was demonstrated in tumor in DL1. At least 72 h of target degradation was observed with every once every three-week doing, a profile that led to robust antitumor activity in MYD88 mutant tumors in preclinical species.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A liquid formulation comprising Compound A, or a pharmaceutically acceptable salt thereof, and a pharmaceutically acceptable excipient and/or carrier;
wherein Compound A is N-[5-(2-hydroxypropan-2-yl)-2-[(1r,4r)-4-{[6-(2-{[2-(2,6-dioxopiperidin-3-yl)-1,3-dioxo-2,3-dihydro-1H-isoindol-4-yl]amino}ethyl)-2-azaspiro[3.3]heptan-2-yl]methyl}cyclohexyl]-1,3benzothiazol-6-yl]-6-(trifluoromethyl)pyridine-2-carboxamide.

2. The liquid formulation of claim 1, comprising Compound A at a concentration of about 0.05%-1.5% % w/w of the total weight of the formulation.

3. The liquid formulation of claim 1, comprising Compound A at a concentration of about 0.5-15 mg/mL.

4. The liquid formulation of claim 1, comprising a solubilizing agent at a concentration of about 10%-50% % w/w of the total weight of the formulation.

5. The liquid formulation of claim 1, comprising a solubilizing agent at a concentration of about 100-500 mg/mL.

6. The liquid formulation of claim 1, comprising a pH modifier at a concentration of about 0.5%-1.5% % w/w of the total weight of the formulation.

7. The liquid formulation of claim 1, comprising a pH modifier at a concentration of about 5-15 mg/mL.

8. The liquid formulation of claim 1, which is at about pH 2 to about pH 6.

9. The liquid formulation of claim 1, which is a unit dosage form, with a volume of from about 10 mL to about 50 mL.

10. A method for treating a relapsed and/or refractory B-cell non-Hodgkin lymphoma in a patient, comprising administering to the patient a therapeutically effective amount of the liquid formulation of claim 1.

11. The method of claim 10, wherein the relapsed and/or refractory B-cell non-Hodgkin lymphoma is selected from diffuse large B-cell lymphoma (DLBCL), active B-cell diffuse large B-cell lymphoma (ABC DLBCL), primary mediastinal B-cell lymphoma, primary extranodal lymphomas, primary CNS lymphoma, primary cutaneous large B-cell lymphoma, follicular lymphoma, chronic lymphocytic leukemia (CLL), small lymphocytic lymphoma (SLL), mantle cell lymphoma (MCL), marginal zone lymphomas, nodal marginal zone B-cell lymphoma, splenic marginal zone B-cell lymphoma, extranodal marginal zone B-cell lymphoma, mucosa-associated lymphoid tissue (MALT) lymphoma, Burkitt lymphoma, Waldenström macroglobulinemia, hairy cell leukemia (HCL), and primary intraocular lymphoma.

12. The method of claim 10, wherein the method comprises administering a dosage of up to about 10.0 mg/kg of Compound A to the patient.

13. (canceled)

14. The method of claim 10, wherein the method comprises administering a dosage of up to about 600 mg of Compound A to the patient.

15. (canceled)

16. The method of claim 10, wherein the method comprises administering Compound A to the patient intravenously.

17. The method of claim 10, wherein the method comprises administering Compound A to the patient once every three weeks (Q3W).

18. The method of claim 17, wherein the method comprises administering Compound A to the patient on day 1 of a 21-day cycle.

19. The method of claim 17, wherein the method comprises administering to a patient about 0.16 mg/kg, about 0.32 mg/kg, about 0.64 mg/kg, about 1.25 mg/kg, about 2.0 mg/kg, about 3.0 mg/kg, about 4.2 mg/kg, or about 5.6 mg/kg of Compound A on day 1 of a 21-day cycle.

20. The method of claim 10, wherein the method comprises administering Compound A to the patient twice every three weeks.

21. The method of claim 20, wherein the method comprises administering Compound A to the patient on day 1 and 2 of a 21-day cycle.

22. The method of claim 21, wherein the method comprises administering to a patient about 0.16 mg/kg, about 0.32 mg/kg, about 0.64 mg/kg, about 1.25 mg/kg, about 2.0 mg/kg, about 3.0 mg/kg, about 4.2 mg/kg, or about 5.6 mg/kg of Compound A on day 1 and day 2 of a 21-day cycle.
